(19) United States
(12) Patent Application Publication
(10) Pub. No.: US 2025/0152697 A1
(43) Pub. Date: May 15, 2025

(54) AUTOLOGOUS CELL BASED SARS-COV-2 VACCINES

(71) Applicant: Orgenesis Inc., Germantown, MD (US)

(72) Inventors: Oren MOSKOVICH, Rishon LeZion (IL); Shlomo SRAGOVICH, Tel Aviv (IL); Irit MEIVAR-LEVY, Yehud (IL)

(73) Assignee: Orgenesis Inc., Germantown, MD (US)

(21) Appl. No.: 18/728,093

(22) PCT Filed: Jan. 12, 2023
(86) PCT No.: PCT/US2023/060541

§ 371 Date: Jul. 11, 2024

Related U.S. Application Data
(60) Provisional application No. 63/299,034, filed on Jan. 13, 2022.

(51) Int. Cl. A61K 39/215; A61K 39/00; A61P 31/14; C12N 5/0784; C12N 7/00; C12N 15/86

(52) U.S. Cl. CPC A61K 39/215; A61P 31/14; C12N 5/0639; C12N 7/00; C12N 15/86; A61K 2039/5154; A61K 2039/572; A61K 2039/70; C12N 2770/20034; C12N 2770/20043

(57) ABSTRACT

Disclosed herein are multi-antigenic autologous, cell-based SARS-CoV-2 vaccines comprising autologous antigen presenting cells (APCs) displaying at least two different SARS-CoV-2 antigens. These vaccines can be used to prevent SARS-CoV-2 infection or COVID-19. Further disclosed are methods for producing and using the vaccines.

### CROSS-REFERENCE TO RELATED APPLICATIONS

This application is a National Phase application of PCT International Application No. PCT/US23/60541, International Filing Date Jan. 12, 2023, claiming the benefit of U.S. Patent Application No. 63/299,034, filed Jan. 13, 2022, which are hereby incorporated by reference.

### FIELD OF DISCLOSURE

Disclosed herein are multi-antigenic autologous, cell-based vaccines comprising autologous antigen presenting cells (APCs) displaying at least two different antigens. Vaccines comprising APCs displaying at least two different SARS-CoV-2 antigens, can be used for preventing SARS-CoV-2 infection or COVID-19. Further disclosed are methods for producing and using the vaccines.

### BACKGROUND

The outbreak of the coronavirus disease 2019 (COVID-19) is caused by a novel coronavirus named Severe Acute Respiratory Syndrome Coronavirus 2 (SARS-CoV-2). A positive-sense, single-stranded RNA virus of the family Coronaviridae. COVID-19 has already claimed the lives of over 6.5 million people worldwide and has affected millions more. Developing effective vaccines and therapies requires understanding how the adaptive immune response recognizes and clears the virus and how the interplay between the virus and the immune system affects the pathology of the disease. The spike (S) glycoprotein of SARS-CoV-2 mediates the entry of the virus into the host cell and is one of the most important antigenic determinants, making it a potential candidate for a vaccine. Current therapeutic armamentarium involves prophylactic vaccine strategies, mainly mRNA- or viral-vector-based, both demonstrating high benefit-to-risk ratio.

Additional structural elements of SARS-CoV-2 include the membrane (M) protein, which is a transmembrane glycoprotein, shaping the virus structure, and the envelope (E) protein, having a key role in the pathogenesis, assembly, and release of the virus. The nucleocapsid (N) protein within the phospholipid bilayer, has a key role in complex formation with viral genome, enhances M protein interaction during assembly, and increases replication of the virus. Importantly, findings suggest that vaccines focused solely on eliciting neutralizing antibodies to the S protein may be insufficient to elicit long-term immunity to coronaviruses.

The conventional methods of vaccine design, involving whole viruses or large proteins may lead to unnecessary antigenic load along with increased chances of side effects, as well as allergenic responses. The S protein is a leading potential target for vaccine design for either SARS-CoV or SARS-CoV-2 infection because of its strong immunogenicity and its roles in virus attachment and cell entry. However, a majority of the mapped T cell responses fall outside of the S protein, therefore raising the possibility that many of the S protein-directed vaccines currently under development may elicit an insufficient CD8+ T cell response. Further, despite current prophylactic vaccine strategies, there is still growing evidence that SARS-CoV-2 mutations, specifically the high mutation burden of the spike protein, are of particular importance (e.g. delta and omicron variants), and present an unmet medical need. Current vaccine effectiveness seems to be short-termed and is reduced against novel SARS-CoV-2 variants, leading to a waned immunity against the virus. Rather than responding to the next outbreak, it is critical to develop a vaccine that would protect against all iterations of coronavirus. Till now, long-term, broad, and effective vaccines have not been developed.

Disclosed herein is a new multi-antigenic autologous cell-based vaccine, comprising antigen presenting cells (APCs). Further disclosed are methods for producing the vaccines, and methods of using them for treating and preventing COVID-19.

### SUMMARY

In one aspect, disclosed herein is multi-antigenic autologous, cell-based SARS-CoV-2 vaccine, said vaccine comprising autologous antigen presenting cells (APCs) displaying at least two different SARS-CoV-2 antigens.

In some related aspects, the APCs are activated.

In some related aspects, at least two different SARS-CoV-2 antigens comprise SARS-CoV-2 structural protein antigens. In some related aspects, at least two different SARS-CoV-2 antigens comprise antigens of the spike (S) protein, membrane (M) protein, nucleocapsid (N) protein, envelope (E) protein, any part thereof, or any combination thereof. In some related aspects, the SARS-CoV-2 spike protein antigen comprises a S1 subunit, S2 subunit, or receptor binding domain (RBD) antigen.

In some related aspects, the APCs comprise dendritic cells, macrophages, B cells, monocytes or a combination thereof. In some related aspects, the APCs comprise dendritic cells.

In some related aspects, the APCs have been contacted with a preparation of SARS-CoV-2 particles ex-vivo. In some related aspects, the preparation of SARS-CoV-2 particles comprises chemically inactivated or attenuated live SARS-CoV-2 particles. In some related aspects, the preparation of SARS-CoV-2 particles comprises at least two SARS-CoV-2 variants.

In some related aspects, the APCs have been contacted with mRNAs encoding SARS-CoV-2 antigens.

In some aspects, the vaccine is produced by a method comprising: (a) obtaining a population of peripheral blood mononuclear cells (PBMCs) from a subject; (b) isolating and culturing the APCs from said population of PBMCs; (c) contacting the APCs with a preparation of SARS-CoV-2 particles for a time period sufficient to generate APCs displaying at least two different SARS-CoV-2 antigens; and (d) optionally isolating and expanding the APCs from step (c).

In some related aspects, disclosed herein is a pharmaceutical composition comprising the vaccine disclosed above.

In some aspects, disclosed herein is a method for production of a multi-antigenic autologous, cell-based SARS-CoV-2 vaccine, comprising autologous antigen presenting cells (APCs) displaying at least two different SARS-CoV-2 antigens, said method comprising: (a) obtaining a population of peripheral blood mononuclear cells (PBMCs) from a subject; (b) isolating and culturing the APCs from said population of PBMCs; (c) contacting the APCs with a preparation of SARS-CoV-2 particles or with mRNAs encoding SARS-CoV-2 antigens for a time period sufficient to generate APCs displaying at least two different SARS-CoV-2 antigens; and (d) optionally isolating and expanding the APCs from step (c).

In some related aspects, the SARS-CoV-2 particles are contacted at a multiplicity of infection (MOI) of between 0.001 and 10. In some related aspects, the MOI is 2.

In some related aspects, the APCs are contacted with a preparation of SARS-CoV-2 particles in the presence of a transduction reagent. In some related aspects, the transduction reagent comprises polybrene (PB). In some related aspects, the transduction reagent comprises Protamine Sulfate.

In some related aspects, disclosed herein is a method for preventing or treating a SARS-CoV-2 infection in a subject, the method comprising: (a) obtaining a population of peripheral blood mononuclear cells (PBMCs) from a subject; (b) isolating and culturing antigen-presenting cells (APCs) from said population of PBMCs; (c) contacting the APCs with a preparation of SARS-CoV-2 particles or with mRNAs encoding SARS-CoV-2 antigens for a time period sufficient to generate APCs displaying at least two different SARS-CoV-2 antigens; (d) optionally isolating and expanding the APCs from step (c); and (e) administering said APCs to said subject.

In some related aspects, the subject is a human. In some related aspects, the subject is a health care worker.

### BRIEF DESCRIPTION OF THE DRAWINGS

The subject matter regarded as multi-antigenic autologous cell-based SARS-CoV-2 vaccine, methods of producing and methods of using thereof, is particularly pointed out and distinctly claimed in the concluding portion of the specification. This vaccine, however, its technical features, and advantages thereof, may best be understood by reference to the following detailed description when read with the accompanying drawings in which:

FIG. 1A shows the relative expression level of SARS-CoV-2 envelope gene in dendritic cells transduced with chemically-inactivated SARS-CoV-2. MOI=multiplicity of infection; PB=polybrene;

FIG. 1B shows the relative expression level of SARS-CoV-2 nucleocapsid gene in dendritic cells transduced with chemically-inactivated SARS-CoV-2. MOI=multiplicity of infection; PB=polybrene;

FIG. 2 shows the study flow of Example 2, wherein T cells were activated using dendritic cells transduced with chemically-inactivated SARS-CoV-2.

FIG. 3 shows naïve T cell activation assessed by flow cytometry following exposure to chemically-inactivated SARS-CoV-2-infected DCs.

FIG. 4 shows the percentage of PMA-activated T cells assessed by flow cytometry following exposure to chemically-inactivated SARS-CoV-2-infected DCs.

FIG. 5 shows the percentage of activated T cells assessed by CD4+/CD137+ marker expression.

FIG. 6 shows the experimental outline of immunogenicity and functional experiments described in Example 3, including DC differentiation and maturation, viral transduction, co-culturing with T cells, and FACS analysis on Days 1, 4, and 7 of the co-culture.

FIG. 7A shows DC morphology after 7 days of differentiation.

FIGS. 7B-7F show representative FACS illustrations of each marker expression: Dio3 (FIG. 7B), CD45 (FIG. 7C), CD86 (FIG. 7D), CD11c (FIG. 7E), HLA-DR (FIG. 7F). A high percentage of the CD45+ cells were also positive for CD86 (82%) (FIG. 7D), CD11c (94%) (FIG. 7E) and HLA-DR (84.5%) (FIG. 7F).

FIGS. 8A-8B showing the results of unpaired Student's t-test analyses for each tested viral gene expression. FIG. 8A for the envelope gene—a significant difference was found between non-transduced, and transduced cells (*p<0.05). FIG. 8B for the nucleocapsid gene a significant difference was found between non-transduced, and transduced cells (**p<0.01).

FIG. 9 schematically showing the gating strategy used to analyze different T cell populations in immunogenicity studies.

FIGS. 10A-10B showing FACS analysis of total PBMCs (FIG. 10A) and T cells (FIG. 10B) on Day 4 (D4) in culture, presenting the expression of CD3, CD4 and CD8 (T cell markers), as well as CD19 (B cell marker).

FIG. 11 One-way ANOVA analysis with Bonferroni's post hoc test was performed (N=4-11 for either non-transduced cells or transduced cells at an MOI=2.5). Main effects were found on Day 1 (F(2,11)=9.549, p=0.00394), and Day 7 (F(2,26)=4.858, p=0.016) for CD4+ cells. In addition, main effects were found on Day 1 (F(2,11)=12.347, p=0.00154), Day 4 (F(2,17)=3.875, p=0.041), and Day 7 (F(2,28)=18.695, p=6.97E-06) for CD8+ cells. On Day 1, significant differences were found between ‘Transduced DCs/T MOI=2.5’ vs. ‘T cell alone’ and ‘DC/T’ groups for CD4+ cells (**p<0.01), as well as ‘Transduced DCs/T MOI=2.5’ vs. ‘T cell alone’ groups for CD8+ cells (**p<0.01). On Day 4, a significant difference was found between ‘Transduced DCs/T MOI=2.5’ vs. ‘T cell alone’ groups for CD8+ cells (*p<0.05). On Day 7, significant differences were found between ‘Transduced DCs/T MOI=2.5’ vs. ‘T cell alone’ groups for CD4+ (**p<0.01), and CD8+ cells (***p<0.001). Additionally, significant differences were found between ‘DC/T’ vs. ‘T cell alone’ and ‘Transduced DCs/T MOI=2.5’ groups (**p<0.01).

FIG. 12 One-way ANOVA analysis with Bonferroni's post hoc test was performed (N=4-11 for either non-transduced cells or transduced cells at an MOI=2.5). Main effects were found on Day 4 (F (2,17)=36.08331, p=7.62248E-07) for CD4+ cells. Additionally, main effects were found on Day 4 (F (2,17)=52.491, p=5.31E-08), and Day 7 (F (2,28)=3.69, p=0.037) for CD8+ cells. On Day 4, significant differences were found between ‘Transduced DCs/T MOI-2.5’ vs. ‘T cell alone’ and ‘DC/T’ groups for CD4+ and CD8+ cells (**p<0.01, ***p<0.001). In addition, on Day 4, for CD8+ cells, a significant difference was found between ‘DC/T’ and ‘T cells alone’ groups (**p<0.01). On Day 7, a significant difference was found between ‘DC/T’ vs. ‘T cell alone’ groups (*p<0.05) for CD8+ cells.

FIG. 13 One-way ANOVA analysis with Bonferroni's post hoc test was performed (N=4-11 for either non-transduced cells or transduced cells at an MOI=2.5). Main effects were found on Day 1 (F (2,10)=21.366, p=0.000245) for CD4+ cells, and on Days 1 (F (2,11)=7.964, p=0.007269), 4 (F (2,20)=5.788, p=0.010387), and 7 (F (2,29)=5.251, p=0.011315) for CD8+ cells. On Day 1, significant differences were found between ‘Transduced DCs/T MOI=2.5’ vs. ‘T cell alone’ groups for CD4+ and CD8+ cells (**p<0.01, ***p<0.001). On Days 4 and 7, significant differences were found between ‘Transduced DCs/T MOI=2.5’ vs. ‘T cell alone’ groups for CD8+ cells only (**p<0.01).

FIG. 14 shows representative FACS illustrations of each marker in T cells co-cultured with SARS-CoV-2-transduced cells (shaded area) or with non-transduced DCs (line).

FIG. 15 One-way ANOVA analysis with Bonferroni's post hoc test was performed (N=4-11 for either non-transduced cells or transduced cells at an MOI=2.5). No significant differences were observed among tested experimental groups.

FIG. 16 One-way ANOVA analysis with Bonferroni's post hoc test was performed (N=4-8 for either non-transduced cells or transduced cells at an MOI=2.5). Main effects were found on Day 1 (F (2,13)=18.499, p=0.000158), Day 4 (F (2,21)=28.460, p=1.049E-06), and Day 7 (F (2,18)=5.85, p=0.011). On Day 1 and Day 4, significant differences were found between ‘T cell alone’ vs. ‘DC/T and Transduced DCs/T MOI=2.5’ groups (**p<0.01, ***p<0.001). On Day 7, a significant difference was observed between ‘T cell alone’ and ‘Transduced DCs/T MOI=2.5’ groups (**p<0.01).

### DETAILED DESCRIPTION

In the following detailed description, numerous specific details and embodiments are set forth in order to provide a thorough understanding of the multi-antigenic cell-based viral vaccine disclosed herein, including methods for producing thereof, and methods for treating and preventing viral infection or disease in a subject by providing the vaccine. In some instances, well-known methods, procedures, and components have not been described in detail so as not to obscure the present disclosure.

In some embodiments, the present disclosure relates to vaccines and to methods for eliciting an immune response to a Coronavirus (e.g. SARS-CoV-2) antigen. An immune response to the Coronavirus target antigen provides a prophylactic or therapeutic effect.

Viral infections can be treated and/or prevented by administering reagents that modulate the immune system. The present vaccines and methods inhibit and/or treat a viral infection (e.g., SARS-CoV-2 infection), and/or ameliorate one or more symptoms associated with the viral infection. The present vaccines and methods are useful in the prophylaxis and/or treatment of a disease caused by coronaviruses (e.g., SARS-CoV-2).

### Multi-Antigenic Cell-Based Viral Vaccines

The present vaccine functions by preparing the immune system to mount a response to a virus. In some embodiments, a vaccine can comprise an antigen, which is a virus or a component of the virus, or a fragment thereof, that is introduced into a subject to be vaccinated in a non-toxic, non-infectious and/or non-pathogenic form. In some embodiments, a vaccine is whole cell-based vaccine comprising antigen presenting cells (APCs) which display viral antigens. In some embodiments, virus antigens include whole live viruses (modified to reduce their virulence) or inactivated viruses, individual viral components (e.g., protein or polysaccharides) and the genetic material of the virus (e.g., RNA or DNA). The antigen in the vaccine causes the subject's immune system to be “primed” or “sensitized” to the virus from which the antigen is derived. Subsequent exposure of the immune system of the subject to the virus results in a rapid, robust and/or specific immune response, that controls or destroys the virus before it can multiply and infect or damage sufficient number of cells in the host organism to cause manifestation of disease symptoms.

In some embodiments, disclosed herein is a multi-antigenic cell-based viral vaccine comprising antigen presenting cells (APCs). In some embodiments, the multi-antigenic cell-based viral vaccine comprises APCs displaying at least two different viral antigens.

A skilled artisan would appreciate that the terms “vaccine” and “immunogenic composition”, may encompass a substance or composition capable of inducing an immune response in a subject. An immune response may include an adaptive immune response (humoral/antibody and/or cellular) inducing memory in an organism, resulting in the generation of antibodies against the foreign invader. The composition may further comprise one or more adjuvants and pharmaceutical carriers.

A skilled artisan would appreciate that a cell-based vaccine comprises APCs that are injected to a subject in need thereof. Commonly used vaccines, usually contain an agent that resembles a disease-causing pathogen, and is often made from a weakened or attenuated form of the pathogen, wherein the injection of the agent induces a specific immune response against the pathogen. Contrarily, APCs or dendritic cell (DC) vaccines, are vaccines comprising cells that present a pathogen's antigens on their cell surface to other types of immune cells and thereby stimulate an immune response against the pathogen. These cells are extracted from the patient's blood, transduced with a polynucleotide encoding the antigen, or contacted with a virus, for a time period sufficient to generate cells displaying an antigen, and then administered back to the patient to stimulate an immune reaction.

In some embodiments, the cell-based vaccine described herein comprises APCs that were contacted (or transduced) with a virus for a time period sufficient to generate APCs displaying a viral antigen. In some embodiments, the cell-based vaccine described herein comprises APCs that were contacted (or transduced) with a preparation of viral particles for a time period sufficient to generate APCs displaying a viral antigen. In some embodiments, the cell-based vaccine described herein comprises APCs that were contacted (or transduced) with mRNA molecules encoding viral antigens for a time period sufficient to generate APCs displaying one or more viral antigens.

In some embodiments, the virus is selected from the group consisting of Severe acute respiratory syndrome coronavirus 2 (SARS-CoV-2), SARS-CoV-1, Middle East respiratory syndrome (MERS), MERS-CoV-1, Adenovirus (ADV), Herpes simplex virus (HSV), Herpes simplex-type 1, Herpes simplex-type 2, Human herpesvirus-type 8, Epstein-Barr virus (EBV), Human cytomegalovirus (CMV), varicella zoster virus (VZV), Human papillomavirus (HPV), Bocavirus (BoV), Hepatitis C Virus (HCV), yellow fever virus, dengue virus, Zika virus, West Nile virus, Japanese encephalitis virus (JEV), polio, Rhinovirus, Ebola virus, Marburg virus, Influenzavirus, Influenzavirus A, Influenzavirus B, Influenzavirus C, Influenzavirus D, Thogotovirus, Quaranjavirus, Measles virus, Parainfluenza virus, Respiratory syncytial virus (RSV), Metapneumovirus (MPV), Rabies virus, Human T-cell lymphotropic virus type 1 (HTLV-1), human immunodeficiency virus (HIV), chikungunya virus and Hepatitis B Virus (HBV).

In some embodiments, the virus is SARS-CoV-2. In some embodiments, the virus is SARS-CoV-1. In some embodiments, the virus is MERS. In some embodiments, the virus is MERS-CoV-1. In some embodiments, the virus is ADV. In some embodiments, the virus is Herpes simplex virus (HSV). In some embodiments, the virus is Herpes simplex-type 1. In some embodiments, the virus is Herpes simplex-type 2. In some embodiments, the virus is Herpes virus-type 8. In some embodiments, the virus is EBV. In some embodiments, the virus is CMV. In some embodiments, the virus is VZV. In some embodiments, the virus is HPV. In some embodiments, the virus is BoV. In some embodiments, the virus is HCV. In some embodiments, the virus is yellow fever virus. In some embodiments, the virus is dengue virus. In some embodiments, the virus is Zika virus. In some embodiments, the virus is West Nile virus. In some embodiments, the virus is Japanese encephalitis virus. In some embodiments, the virus is polio virus. In some embodiments, the virus is Rhinovirus. In some embodiments, the virus is Ebola virus. In some embodiments, the virus is Marburg virus. In some embodiments, the virus is Influenzavirus. In some embodiments, the virus is Influenzavirus A. In some embodiments, the virus is Influenzavirus B. In some embodiments, the virus is Influenzavirus C. In some embodiments, the virus is Influenzavirus D. In some embodiments, the virus is Thogotovirus. In some embodiments, the virus is Quaranjavirus. In some embodiments, the virus is Measles virus. In some embodiments, the virus is Parainfluenza virus. In some embodiments, the virus is RSV. In some embodiments, the virus is MPV. In some embodiments, the virus is rabies virus. In some embodiments, the virus is HTLV-1. In some embodiments, the virus is HIV. In some embodiments, the virus is chikungunya virus. In some embodiments, the virus is HBV. In some embodiments, the virus is associated with human cancer. In some embodiments, the virus is a disease-causing virus.

In some embodiments, the virus comprises a Baltimore classification Group I virus of double-stranded DNA viruses (e.g. Adenoviruses, Herpesviruses including Epstein-Barr virus, Poxviruses, Polyoma viruses including BK virus and JC virus (human polyomavirus 2)). In some embodiments, the virus comprises a Baltimore classification Group II virus of single-stranded (or “sense”) DNA viruses (e.g. Parvoviruses). In some embodiments, the virus comprises a Baltimore classification Group III virus of double-stranded RNA viruses (e.g. Reoviruses). In some embodiments, the virus comprises a Baltimore classification Group IV virus of single-stranded (sense) RNA viruses (e.g. Picornaviruses, Togaviruses, Coronavirus including SARS-CoV-2). In some embodiments, the virus comprises a Baltimore classification Group V virus of single-stranded (antisense) RNA viruses (e.g. Orthomyxoviruses, Rhabdoviruses). In some embodiments, the virus comprises a Baltimore classification Group VI virus of single-stranded (sense) RNA viruses with DNA intermediate in life-cycle (e.g. Retroviruses). In some embodiments, the virus comprises a Baltimore classification Group VII virus of double-stranded DNA viruses with RNA intermediate in life-cycle (e.g. Hepadnaviruses).

A skilled artisan would appreciate that the terms “multi-antigenic”, “multi-antigenic APCs” and “multi-antigen vaccine” may encompass more than one antigen, for example at least two, at least three, or at least four antigens. Thus, in some embodiments, an APC displays two different viral antigens. In some other embodiments, the APC displays three different viral antigens. In some embodiments, the multi-antigenic APC displays different antigens of a single viral protein. In some embodiments, the vaccine disclosed herein is pan-antigenic. In some embodiments, the vaccine disclosed herein comprises a number of antigens.

A skilled artisan would appreciate that the term “different antigen” may encompass antigens that are distinct from each other. In some embodiments, different antigens of a single viral protein, represent distinct antigens displayed on the surface of an APC after the viral protein antigen has been processed by the APC into small fragments, or peptides. In some embodiments, the APCs display two or more different virus antigens, including 2, 3, 4, 5, 6, 7, 8, 9, 10 or more antigens. In some embodiments, the APCs display at least one viral antigen. In some embodiments, the APCs display at least two different viral antigens. In some embodiments, the vaccine further comprises APCs ectopically expressing at least one viral antigen.

A skilled artisan would appreciate that the term “antigen” may encompass a molecule containing one or more epitopes (either linear, conformational or both) or immunogenic determinants that will stimulate a host's immune-system, such as a mammal's immune system, to make a humoral and/or cellular antigen-specific response. In some embodiments, the term antigen refers to a molecule or molecular structure that can be bound by an antigen-specific antibody or B cell antigen receptor. The term is used interchangeably with the term “immunogen.” An antigen may be a virus (e.g., an inactivated virus, or an attenuated virus), a whole protein, a truncated protein, a fragment of a protein or a peptide. Antigens may be naturally occurring, genetically engineered variants of the protein, or may be codon optimized. Furthermore, an “antigen” refers to a protein which includes modifications, such as deletions, additions and substitutions, generally conservative in nature, to the naturally occurring sequence, so long as the protein maintains the ability to elicit an immunological response, as defined herein. These modifications may be deliberate, as through site-directed mutagenesis, or may be accidental, such as through mutations of hosts which produce the antigens. Antigens of the present disclosure may also be codon optimized by methods known in the art to improve their expression or immunogenicity in the host.

In some embodiments, the antigen is immunogenic. In some embodiments, the antigen comprises a protein-based antigen. In some embodiments, the antigen comprises a peptide-based antigen. In some embodiments, the antigen comprises a viral antigen. A “viral antigen,” as used herein, is an antigen from a virus and includes, but is not limited to, a SARS-CoV-2 antigen.

In some embodiments, the APCs display at least two different structural protein antigens. In some embodiments, the APCs display at least two different viral antigens comprising antigens of the spike (S) protein, membrane (M) protein, nucleocapsid (N) protein, envelope (E) protein, glycoprotein (G), any part thereof, or any combination thereof. In some embodiments, the APCs display at least two different viral antigens comprising antigens of the spike (S) protein or part thereof. In some embodiments, the APCs display at least two different viral antigens comprising antigens of the membrane (M) protein or part thereof. In some embodiments, the APCs display at least two different viral antigens comprising antigens of the nucleocapsid (N) protein or part thereof. In some embodiments, the APCs display at least two different viral antigens comprising antigens of the envelope (E) protein or part thereof. In some embodiments, the APCs display at least two different viral antigens comprising antigens of the glycoprotein (G) or part thereof. In some embodiments, the APCs display at least two different viral antigens comprising antigens of the receptor binding domain (RBD) or part thereof.

In some embodiments, the multi-antigenic cell-based viral vaccine described herein comprises dendritic cells, macrophages, B cells, monocytes or a combination thereof. In some embodiments, the multi-antigenic cell-based viral vaccine described herein comprises APCs. In some embodiments, APCs comprise dendritic cells, macrophages, B cells, monocytes or a combination thereof. In some embodiments, the multi-antigenic cell-based viral vaccine comprises dendritic cells (DCs). In some embodiments, the multi-antigenic cell-based viral vaccine comprises macrophages. In some embodiments, the multi-antigenic cell-based viral vaccine comprises B cells. In some embodiments, the multi-antigenic cell-based viral vaccine comprises monocytes.

In some embodiments, the APCs comprise autologous APCs. A skilled artisan would appreciate that the term “autologous” refers to cells obtained from the same individual to which the cells are administered. In some embodiments, the APCs comprise allogeneic APCs. A skilled artisan would appreciate that the term “allogeneic” may encompass cells that are derived from separate individuals of the same species. In some embodiments, allogeneic donor cells are genetically distinct from the recipient. In some embodiments, the APCs comprise syngeneic APCs. A skilled artisan would appreciate that the term “syngeneic” may encompass cells that are genetically similar or identical and hence immunologically compatible. In some embodiments, syngeneic cells are so closely related that transplantation does not provoke an immune response.

In some embodiments, the present vaccines and methods relate to whole cell-based vaccines. In some embodiments, the present whole cell vaccines provide multiple antigens that can be targeted by both the innate and adaptive immune systems. In some embodiments, the present whole-cell-based vaccine serves as an adjuvant on its own, because of its ability to stimulate the immune system in a non-specific manner. In some embodiments, the present whole-cell vaccine comprises allogeneic cells providing MHC-allotypes (alternative histocompatibility complexes), which are powerful stimulators of the immune response. In some embodiments, the present vaccine comprises a whole cell vaccine, e.g., an allogeneic or autologous whole cell vaccine. In some embodiments, the present whole-cell vaccine comprises syngeneic cells.

A skilled artisan would appreciate that APCs, which in some embodiments are also termed accessory cells, are cells that display antigens complexed with major histocompatibility complexes (MHCs) on their surface, thus presenting the antigens to T cells which recognize these complexes using their T cell receptors (TCRs). In some embodiments, APCs present foreign antigens to T cells. In some embodiments, APCs present foreign antigens to helper T cells. In some embodiments, APCs present (or display) foreign antigens to cytotoxic T cells (CTLs).

A skilled artisan would appreciate that many cell types can present antigens. In some embodiments, any of these cells can be used in the vaccines disclosed herein. These cells can be found in a variety of tissue types. In some embodiments, APCs comprise professional antigen-presenting cells. In some embodiments, the terms “antigen presenting cells”, “APCs” and “professional antigen-presenting cells”, are used herein interchangeably, having all the same features and limitations. In some embodiments, APCs comprise dendritic cells, macrophages, B cells, monocytes or a combination thereof. In some embodiments, APCs comprise dendritic cells (DCs). In some embodiments, APCs comprise macrophages. In some embodiments, APCs comprise B cells. In some embodiments, APCs comprise monocytes. In some embodiments, APCs comprise a combination of two or more types of APCs. In some embodiments, APCs comprise activated APCs.

In some embodiments, the APCs are loaded with one or more virus antigens (e.g., SARS-CoV-2). A cell “loaded” or “pulsed” with a polynucleotide (such as mRNA), peptide or protein means that the cell has been incubated or contacted with the polynucleotide, peptide or protein under conditions permitting entry into, and/or attachment onto the cell. For example, APCs (e.g., dendritic cells) can be incubated with one or more virus antigens (e.g., Coronavirus antigens) or viral particles under conditions that are needed to load the MHC of the APC (e.g., the dendritic cell). Suitable conditions for antigen loading are provided, that permit an APC to contact, process and/or present one or more antigens on its MHC, whether intracellularly or on the cell surface.

In some embodiments, the APCs are contacted with mRNAs encoding viral antigens. The mRNAs enter the APCs where they are translated into viral antigen proteins or peptides and are presented on the APC surface. The term “polynucleotide” encompasses a single or double stranded nucleic acid sequence in the form of an RNA sequence, such as messenger RNA (mRNA), a complementary polynucleotide sequence (cDNA), a genomic polynucleotide sequence and/or a composite polynucleotide sequence (e.g., a combination of the above). A polynucleotide may be linear or branched, and optionally contains synthetic, non-natural, or altered nucleotide bases.

A skilled artisan would appreciate that an APC loaded with a preparation of viral particles, having multiple antigens, can take up antigens, process them, and display (or present) antigens on their surface, along with the MHC. A skilled artisan would appreciate that the terms “contacting APCs”, “transducing APCs” and “incubating APCs” may encompass the loading of APCs with antigens, which in some embodiments, may be preparations of viral particles.

In some embodiments, the APCs are permissive cells. In some embodiments, the APCs are infection-permissive cells. In some embodiments, the APCs are transfection-permissive cells. In some embodiments, the APCs are transduction-permissive cells. In some embodiments, the permissive cells are murine cells. In some embodiments, the permissive cells are humanized murine cells. In some embodiments, the permissive cells are primate cells. In some embodiments, the permissive cells are human cells. In some embodiments, the permissive cells are human autologous cells. In some embodiments, the permissive cells are human non-autologous cells. In some embodiments, the permissive cells are a human patient's cells. In some embodiments, the permissive cells are white blood cells. In some embodiments, the permissive cells are T cells. In some embodiments, the permissive cells are B cells. In some embodiments, the permissive cells are macrophage cells. In some embodiments, the permissive cells are dendritic cells. In some embodiments, the permissive cells are antigen-presenting cells. In some embodiments, the permissive cells are stem cells.

DCs were found to serve as an ideal choice in the case of antitumor vaccines due to the unique characteristics these cells pose. DCs are considered the most effective APCs responsible for primarily sensitizing naive T cells to specific antigens. Compared with B cells and monocytes, DCs are 10- to 100-times more potent in inducing T-cell proliferation, playing an important role in the establishment of immunologic memory. Importantly, DCs are able to use soluble protein antigens to sensitize both CD4+ and CD8+ T cells inducing antigen-specific cytotoxic T lymphocytes (CTLs), hence contrasting monocytes and B cells. Given this capability, a wider range of potential antigen targets can be effectively used to sensitize T cells by developing DC-based vaccines.

Furthermore, as the principal APC, DCs have the advantageous ability to cross-prime. Specifically, DCs can present via the classical pathways of presenting exogenous antigens on MHC class II molecules and endogenous antigens on MHC class I molecules. Additionally, DCs can also route exogenous antigens into the pathway that is intended for presenting on MHC class I molecules, as well as necessary for the generation of CTLs. The presentation capability of exogenous tumor antigens on MHC class I molecules has been shown in different in vitro and in vivo models. Although B cells and macrophages have also demonstrated an ability to cross-prime to some extent, they do so less efficiently, compared with DCs.

A skilled artisan would appreciate, that in the circulation and in peripheral tissues, DCs are generally found in an immature form. As soon as the cells receive signals of maturation, DCs display an increased expression of surface MHC molecules to enhance antigen presentation. Additionally, DCs upregulate chemokine receptors to facilitate migration to nearby lymph nodes, as well as costimulatory molecules necessary for amplification of the T-cell response. The maturation signals include inflammatory signals from the local microenvironment (e.g. TNF, IL-1, and prostaglandins), T cell-associated signals, and pathogen-related molecules (e.g. LPS, Bacterial DNA, and dsDNA). Based on the type of maturation signals received by DCs, the cells can mature into various phenotypes, thereby affecting cytokine secretion and T-cell interactions.

A variety of precursors contribute to the development of DCs, therefore leading to different DC subsets, the choice of which has an importance in vaccination. Selecting the right DC subset for a vaccine should take into consideration logistic terms, such as obtaining an adequate number of cells, as well as biological factors, as how these DCs differentiate, mature and function as part of the immune response. Various DC lineages were investigated in mouse models, examining the immune responses induced by the different cells. Interestingly, the ability to cross-prime varied among the murine lineages, with specific cross-presentation exhibited in DCs of the CD8+ lineage. This suggests that similarly to the functional variability in mice, distinctive capabilities of human DCs derived from different lineages may also be observed.

According to evidence in humans, cross-presentation, being an essential part of mediating CD8+ immunity to exogenous antigens, does not occur in all DC subsets. In this respect, monocyte-derived DCs (moDCs) have demonstrated an ability to cross-present, similarly to several other tissue DCs. Yet, DC receptors are also important for cross-presentation, and manipulation of these receptors to enhance this function are a subject of current studies.

A skilled artisan would appreciate that the role of the DC as a potent and versatile APC, coupled with the capability of this cell type as an immune effector cell, makes the DC an ideal vehicle for a vaccine. Nevertheless, DCs regulate the nature of the subsequent immune response, and not only prime naive T cells for antigen recognition. As mentioned above, these variable responses relate to the heterogeneous cell lineage. Various approaches can be employed when exploiting DCs for vaccination including: 1) non-targeted peptide/protein and nucleic acid-based vaccines captured by DCs in vivo; 2) vaccines composed of antigens directly coupled to anti-DC antibodies; 3) vaccines composed of ex vivo generated DCs that are loaded with antigens. All these approaches can be applied to produce the vaccines disclosed herein.

In recent years, the understanding of DC biology has increased substantially, as well as the finding of different subsets of DCs with specific functions, coupled with distinct molecular mechanisms employed by the cells to regulate the immune response. Given that DCs have a capacity to capture, process, and present antigens to T cells, DCs serve as an essential component of vaccination, with adjuvants that act by inducing DC maturation as supplementary vaccine components. Additionally, resident DCs in lymph-nodes can be reached by vaccines through the lymphatic system, thus exhibiting another characteristic of DC-based vaccines. The efficient presentation of antigens to T cells, is a common feature and a critical step in vaccination. Thus, taking advantage of the DC subset diversity, as well as plasticity, together with the fact that these cells are the most efficient APCs, produces improved therapeutic vaccines.

In some embodiments, a DC comprises conventional dendritic cell (cDC) or myeloid dendritic cell (mDC). In some embodiments, a DC comprises plasmacytoid dendritic cell (pDC). In some embodiments, DCs comprise CD1c+ myeloid DCs. In some embodiments, DCs comprise CD141+ myeloid DCs. In some embodiments, DCs comprise CD303+ plasmacytoid DCs. In some embodiments, DCs comprise a combination of two or more types of DCs.

DC and macrophage cell populations possess different mechanisms for invoking an immune response. Therefore, using both cell types in a dual provides a complementary effect that elicits a broader and more effective immune response than a vaccination/treatment with either DC or macrophages alone. Furthermore, a key benefit of this dual approach is that a multi-epitope peptide-driven vaccine in the absence of viral genome and various glycan-conjugated antigens may elicit strong and targeted immune responses, as well as minimize the side effects of vaccination, e.g. cytokine release syndrome, hence improving vaccine design. Accordingly, in some embodiments the vaccine disclosed herein in detail comprises DC and macrophage cell populations.

In some embodiments, APCs have been contacted with a preparation of viral particles ex-vivo. In some embodiments, APCs have been transduced with a preparation of viral particles ex-vivo. In some embodiments, APCs comprise activated APCs. In some embodiments, APCs have been contacted with a preparation of viral particles ex-vivo for a time period sufficient to generate APCs displaying at least two different viral antigens.

In some embodiments, the preparation of viral particles comprises one or more inactivated (or attenuated) viruses. In some embodiments, the preparation of viral particles comprises one or more inactivated viruses. In some embodiments, the preparation of viral particles comprises one or more attenuated viruses. In some embodiments, the preparation of viral particles comprises chemically inactivated or attenuated live particles. In some embodiments, the preparation of viral particles comprises chemically inactivated particles. In some embodiments, the preparation of viral particles comprises attenuated live particles. In some embodiments, the preparation of viral particles comprises modified particles. In some embodiments, the preparation of viral particles comprises genetically modified particles. In some embodiments, the preparation of viral particles comprises at least two virus variants. In some embodiments, the vaccine comprises more than one APC population. In some embodiments, the vaccine comprises numerous APC populations, each APC population loaded with a different virus variant.

An artisan would appreciate that the term “variant” may encompass the viral strains of a virus, e.g. delta and omicron variants of SARS-CoV-2, which arise from mutations or groups of mutations.

Viruses (e.g., Coronavirus) may be inactivated using a physical and/or chemical method. Viruses (e.g., Coronavirus) can be inactivated by gamma irradiation, UV irradiation, or other methods known in the art. A range of inactivation agents or methods have been described to inactivate viruses for vaccine purposes. Examples of viral inactivation methods include, gamma irradiation (Martin et al. Vaccine 28 (18): 3143-3151 (2010b)); Alsharifi and Mullbacher Immunol Cell Biol 88 (2): 103-104 (2010)), UV treatment (Budowsky et al. Arch Virol 68 (3-4): 239-247 (1981)), heat (Nims and Plavsic, N Engl J Med 352 (14): 1411-1412 (2012)), ascorbic acid (Madhusudana et al. Int J Infect Dis 8 (1): 21-25 (2004)), ethylenimine derivatives (Larghi and Nebel, J Clin Microbiol 11 (2): 120-122 (1980)), psorlens (Maves et al. Vaccine 29 (15): 2691-2696 (2011)), hydrogen peroxide (Amanna et al. Nat Med 18 (6): 974-979 (2012)), and other methods (Stauffer et al. Recent Pat Antiinfect Drug Discov 1 (3): 291-296 (2006)). Sanders et al. provide a detailed discussion of inactivated viral vaccines in Vaccine Analysis: Strategies, Principles, and Control, Chapter 2. Springer, 2014.

In some embodiments, a virus can be inactivated chemically. In some embodiments, a virus can be inactivated by gamma irradiation. A person skilled in the art would recognize that the suitable irradiation dose to inactivate a virus in cells may vary upon the virus, specific viral strain (or variant), number of cells carrying a virus, etc. In one embodiment, the gamma irradiation dose for inactivation of a virus is about 25-40 kGy. In another embodiment, a virus can be inactivated using UV irradiation. In another embodiment, UV irradiation dose of 230-280 nm at energies 900-1000 Joule/m2 is used for the inactivation of a virus. In one embodiment, the regimen used for inactivation of a virus in infected cells also renders the cells proliferation incompetent.

A skilled artisan would appreciate that the terms “attenuation” and “attenuated” may encompass a virus that is modified to reduce toxicity to a host. The host can be a human or animal host, or an organ, tissue, or cell. The virus can be attenuated to reduce binding to a host cell, to reduce spread from one host cell to another host cell, or to reduce intracellular growth in a host cell. Attenuation can be assessed by measuring, e.g., an indicator of toxicity, the LD₅₀, the rate of clearance from an organ, or the competitive index (see, e.g., Auerbuch, et al. (2001) Infect. Immunity 69:5953-5957). Generally, an attenuation results in an increase in the LD₅₀ by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold.

In some embodiments, APCs have been contacted with mRNAs encoding viral antigens. In some embodiments, APCs have been contacted with mRNAs encoding viral antigens ex-vivo. In some embodiments, APCs have been contacted with mRNAs encoding viral antigens for a time period sufficient to generate APCs displaying at least two different viral antigens. In some embodiments, APCs have been contacted with mRNAs encoding viral antigens ex-vivo for a time period sufficient to generate APCs displaying at least two different viral antigens.

In some embodiments, the mRNAs encode structural protein antigens. In some embodiments, the mRNAs encode viral antigens comprising antigens of the spike (S) protein, membrane (M) protein, nucleocapsid (N) protein, envelope (E) protein, glycoprotein (G), any part thereof, or any combination thereof. In some embodiments, the mRNAs encode viral antigens comprising antigens of the spike (S) protein or part thereof.

In some embodiments, the mRNAs encode viral antigens comprising antigens of the membrane (M) protein or part thereof. In some embodiments, the mRNAs encode viral antigens comprising antigens of the nucleocapsid (N) protein or part thereof. In some embodiments, the mRNAs encode viral antigens comprising antigens of the envelope (E) protein or part thereof. In some embodiments, the mRNAs encode viral antigens comprising antigens of the glycoprotein (G) or part thereof. In some embodiments, the mRNAs encode viral antigens comprising antigens of the receptor binding domain (RBD) or part thereof.

In some embodiments, the APCs disclosed herein express a cytokine. In some embodiments, the APCs disclosed herein ectopically express a cytokine. In some embodiments, the cytokine is selected from the group consisting of granulocyte macrophage colony-stimulating factor (GM-CSF), interferon alpha (IFN-α), interleukin-2 (IL-2), interleukin-12 (IL-12), tumor necrosis factor alpha (TNF-α), and any combination thereof. In some embodiments, the APCs express GM-CSF. In some embodiments, the APCs express IFN-α. In some embodiments, the APCs express IL-2. In some embodiments, the APCs express IL-12. In some embodiments, the APCs express TNF-α.

In some embodiments, the cytokine is substantially similar to the human form of the protein or is derived from the protein of the human sequence (i.e., of human origin). In some embodiments, cytokines of other mammals with substantial homology to the human forms of IL-2, GM-CSF, TNF-α, and others, may be used in the present composition or method when demonstrated to exhibit similar activity on the immune system. In some embodiments, the present composition or method uses proteins that are substantially analogous to any particular cytokine but have relatively minor changes of protein sequence.

In some embodiments, the APCs disclosed herein ectopically express CD40L, CD80, 4-1BBL, CD40, and MBL2, or any combination thereof. In some embodiments, the APCs express CD40L. In some embodiments, the APCs express CD80. In some embodiments, the APCs express 4-1BBL. In some embodiments, the APCs express CD40. In some embodiments, the APCs express MBL2.

A skilled artisan would appreciate that the term “ectopic” may encompass a process which occurs out of its natural place. Thus, ectopic expression of a polypeptide in a cell comprises the expression of the polypeptide in a cell which would not naturally express it. In some embodiments, the terms “ectopic”, “exogenous”, or “artificial” can be used interchangeably, having all the same meaning and limitations.

In some embodiments, the cytokine, CD40L, CD80, 4-1BBL, CD40, or MBL2 are encoded by a vector transduced to the APCs. In some embodiments, the vector comprises a viral vector or a non-viral vector. In some embodiments, the vector comprises a viral vector. In some embodiments, the vector comprises a non-viral vector.

In some embodiments, the APCs disclosed herein may be genetically engineered using any vector to express a cytokine.

A skilled artisan would appreciate that the term “vector” may encompass a polynucleotide capable of transporting another nucleic acid to which it has been linked. The present vectors can be, for example, a plasmid vector, a single- or double-stranded phage vector, or a single- or double-stranded RNA or DNA viral vector. Such vectors include, but are not limited to, chromosomal, episomal and virus-derived vectors, e.g., vectors derived from bacterial plasmids, bacteriophages, yeast episomes, yeast chromosomal elements, and viruses such as baculoviruses, papova viruses, SV40, vaccinia viruses, adenoviruses, fowl pox viruses, pseudorabies viruses and retroviruses, and vectors derived from combinations thereof, such as those derived from plasmid and bacteriophage genetic elements, cosmids and phagemids.

Expression vectors can be used to replicate and/or express the nucleotide sequence encoding, e.g., a virus antigen, and/or a cytokine in a cell (e.g., a mammalian cell such as a human cell). A variety of expression vectors useful for introducing into cells the polynucleotides described herein are well known in the art. Recombinant vectors are prepared using standard techniques known in the art and contain suitable control elements operably linked to the nucleotide sequence encoding the target antigen.

In some embodiments, a vector comprising a nucleic acid sequence encoding a cytokine (and/or a nucleic acid sequence encoding a virus antigen) may be transduced to a cell in vitro, using any of a number of methods known in the art, which include electroporation, membrane fusion with liposomes, Lipofectamine treatment, incubation with calcium phosphate-DNA precipitate, DEAE-dextran mediated transfection, infection with modified viral nucleic acids, direct microinjection into single cells, etc. Procedures for the cloning and expression of modified forms of a native protein using recombinant DNA technology are generally known in the art, as described in Ausubel, et al., 1992 and Sambrook, et al., 1989.

In some embodiments, the vaccine described herein in detail is produced by a method comprising:
a) obtaining a population of peripheral blood mononuclear cells (PBMCs) from a subject;
b) isolating and culturing the APCs from the population of PBMCs;
c) contacting the APCs with a preparation of viral particles for a time period sufficient to generate APCs displaying at least two different virus antigens; and
d) optionally isolating and expanding the APCs from step (c).

In some embodiments, the vaccine described herein in detail is produced by a method comprising:
(a) obtaining a population of peripheral blood mononuclear cells (PBMCs) from a subject;
(b) isolating and culturing the APCs from the population of PBMCs;
(c) contacting the APCs with mRNAs encoding viral antigens for a time period sufficient to generate APCs displaying at least two different viral antigens; and
(d) optionally isolating and expanding the APCs from step (c).

### APCs Displaying SARS-CoV-2 Antigens

Severe acute respiratory syndrome coronavirus 2 (SARS-CoV-2) is the strain of coronavirus that causes coronavirus disease 2019 (COVID-19), the respiratory illness responsible for the COVID-19 pandemic. In some embodiments, the terms “SARS-CoV-2”, “coronavirus” “2019 novel coronavirus”, “2019-nCoV”, “human coronavirus 2019”, and “HCoV-19” can be used interchangeably, having all the same meaning and limitations.

In some embodiments, disclosed herein is a multi-antigenic cell-based SARS-CoV-2 vaccine comprising antigen presenting cells (APCs). In some embodiments, the multi-antigenic cell-based SARS-CoV-2 vaccine comprises APCs displaying at least two different SARS-CoV-2 antigens. In some embodiments, the vaccine comprises all antigens present in SARS-CoV-2. In some embodiments, the vaccine comprises least two different SARS-CoV-2 structural protein antigens. In some embodiments, the vaccine comprises a multi-antigenic autologous cell-based SARS-CoV-2 vaccine.

In some embodiments, the APCs comprise autologous APCs. In some embodiments, the APCs comprise allogeneic APCs. In some embodiments, the APCs comprise syngeneic APCs.

In some embodiments, the cell-based vaccine described herein comprises APCs that were contacted (or transduced) with a preparation of SARS-CoV-2 particles for a time period sufficient to generate APCs displaying a viral antigen.

In some embodiments, the cell-based vaccine described herein comprises APCs that have been contacted with mRNAs encoding SARS-CoV-2 antigens. In some embodiments, the cell-based vaccine described herein comprises APCs that have been contacted with mRNAs encoding SARS-CoV-2 antigens for a time period sufficient to generate APCs displaying at least two different SARS-CoV-2 antigens.

In some embodiments, the multi-antigenic autologous, cell-based SARS-CoV-2 vaccine comprises dendritic cells, macrophages, B cells, monocytes or a combination thereof. In some embodiments, the multi-antigenic autologous, cell-based SARS-CoV-2 vaccine comprises dendritic cells (DCs). In some embodiments, the multi-antigenic autologous, cell-based SARS-CoV-2 vaccine comprises macrophages. In some embodiments, the multi-antigenic autologous, cell-based SARS-CoV-2 vaccine comprises B cells. In some embodiments, the multi-antigenic autologous, cell-based SARS-CoV-2 vaccine comprises monocytes.

In some embodiments, the APC displays two different SARS-CoV-2 antigens. In some other embodiments, the APC displays three different antigens. In some embodiments, the multi-antigenic APC displays different antigens of a single SARS-CoV-2 protein, such as two different spike (S) protein antigens. In some embodiments, the multi-antigenic APC may display antigens of different SARS-CoV-2 proteins, such as spike (S) protein, membrane (M) protein, nucleocapsid (N) protein, and envelope (E) protein.

In some embodiments, the APCs display two or more different SARS-CoV-2 antigens, including 2, 3, 4, 5, 6, 7, 8, 9, 10 or more antigens. In some embodiments, the APCs display at least 2 different SARS-CoV-2 antigens. In some embodiments, the APCs display 2 different SARS-CoV-2 antigens. In some embodiments, the APCs display 3 different SARS-CoV-2 antigens. In some embodiments, the APCs display 4 different SARS-CoV-2 antigens. In some embodiments, the APCs display 5 different SARS-CoV-2 antigens. In some embodiments, the APCs display 6 different SARS-CoV-2 antigens. In some embodiments, the APCs display 7 different SARS-CoV-2 antigens. In some embodiments, the APCs display 8 different SARS-CoV-2 antigens. In some embodiments, the APCs display 9 different SARS-CoV-2 antigens. In some embodiments, the APCs display 10 different SARS-CoV-2 antigens. In some embodiments, the APCs display at least 10 different SARS-CoV-2 antigens.

In some embodiments, APCs displaying at least two SARS-CoV-2 antigens (e.g., comprising at least one inactivated or attenuated virus particle) can be modified to express one or more cytokines, and administered to an uninfected subject, to serve as a vaccine and elicit an enhanced immune response to confer the ability to resist subsequent infection by the virus.

In some embodiments, APCs have been contacted with a preparation of SARS-CoV-2 particles ex-vivo. In some embodiments, APCs have been transduced with a preparation of SARS-CoV-2 particles ex-vivo. In some embodiments, APCs comprise activated APCs. In some embodiments, APCs have been contacted with a preparation of SARS-CoV-2 particles for a time period sufficient to generate APCs displaying at least two different SARS-CoV-2 antigens. In some embodiments, APCs have been contacted with a preparation of SARS-CoV-2 particles ex-vivo for a time period sufficient to generate APCs displaying at least two different SARS-CoV-2 antigens.

In some embodiments, the preparation of SARS-CoV-2 particles comprises one or more inactivated (or attenuated) SARS-CoV-2 viruses. In some embodiments, the preparation of SARS-CoV-2 particles comprises one or more inactivated SARS-CoV-2 viruses. In some embodiments, the preparation of SARS-CoV-2 particles comprises one or more attenuated SARS-CoV-2 viruses. In some embodiments, the preparation of SARS-CoV-2 particles comprises chemically inactivated (ciSARS-CoV-2) or attenuated live SARS-CoV-2 particles. In some embodiments, the preparation of SARS-CoV-2 particles comprises chemically inactivated SARS-CoV-2 particles. In some embodiments, the preparation of SARS-CoV-2 particles comprises attenuated live SARS-CoV-2 particles. In some embodiments, the preparation of SARS-CoV-2 particles comprises modified SARS-CoV-2 particles. In some embodiments, the preparation of SARS-CoV-2 particles comprises genetically modified SARS-CoV-2 particles.

In some embodiments, the preparation of SARS-CoV-2 particles comprises at least one SARS-CoV-2 variant. In some embodiments, the preparation of SARS-CoV-2 particles comprises two or more different SARS-CoV-2 variants, including 2, 3, 4, 5, 6, 7, 8, 9, 10 or more variants. In some embodiments, the preparation of SARS-CoV-2 particles comprises at least 2 different SARS-CoV-2 variants. In some embodiments, the preparation of SARS-CoV-2 particles comprises 2 different SARS-CoV-2 variants. In some embodiments, the preparation of SARS-CoV-2 particles comprises 3 different SARS-CoV-2 variants. In some embodiments, the preparation of SARS-CoV-2 particles comprises 4 different SARS-CoV-2 variants. In some embodiments, the preparation of SARS-CoV-2 particles comprises 5 different SARS-CoV-2 variants. In some embodiments, the preparation of SARS-CoV-2 particles comprises 6 different SARS-CoV-2 variants. In some embodiments, the preparation of SARS-CoV-2 particles comprises 7 different SARS-CoV-2 variants. In some embodiments, the preparation of SARS-CoV-2 particles comprises 8 different SARS-CoV-2 variants. In some embodiments, the preparation of SARS-CoV-2 particles comprises 9 different SARS-CoV-2 variants. In some embodiments, the preparation of SARS-CoV-2 particles comprises 10 different SARS-CoV-2 variants. In some embodiments, the preparation of SARS-CoV-2 particles comprises at least 10 different SARS-CoV-2 variants.

In some embodiments, the preparation of SARS-CoV-2 particles comprises Alpha variant particles. In some embodiments, the preparation of SARS-CoV-2 particles comprises Beta variant particles. In some embodiments, the preparation of SARS-CoV-2 particles comprises Gamma variant particles. In some embodiments, the preparation of SARS-CoV-2 particles comprises Delta variant particles. In some embodiments, the preparation of SARS-CoV-2 particles comprises Omicron variant particles. In some embodiments, the preparation of SARS-CoV-2 particles comprises Alpha, Beta, Gamma, Delta, or Omicron variant particles, or any combination thereof.

In some embodiments, APCs have been contacted with mRNAs encoding SARS-CoV-2 antigens. In some embodiments, APCs have been contacted with mRNAs encoding SARS-CoV-2 antigens ex-vivo. In some embodiments, APCs have been contacted with mRNAs encoding SARS-CoV-2 antigens for a time period sufficient to generate APCs displaying at least two different SARS-CoV-2 antigens. In some embodiments, APCs have been contacted with mRNAs encoding SARS-CoV-2 antigens ex-vivo for a time period sufficient to generate APCs displaying at least two different SARS-CoV-2 antigens.

In some embodiments, the mRNAs encode antigens of at least one SARS-CoV-2 variant. In some embodiments, the mRNAs encode antigens of two or more different SARS-CoV-2 variants, including 2, 3, 4, 5, 6, 7, 8, 9, 10 or more variants. In some embodiments, the mRNAs encode antigens of at least 2 different SARS-CoV-2 variants. In some embodiments, the mRNAs encode antigens of 2 different SARS-CoV-2 variants. In some embodiments, the mRNAs encode antigens of 3 different SARS-CoV-2 variants. In some embodiments, the mRNAs encode antigens of 4 different SARS-CoV-2 variants. In some embodiments, the mRNAs encode antigens of 5 different SARS-CoV-2 variants. In some embodiments, the mRNAs encode antigens of 6 different SARS-CoV-2 variants. In some embodiments, the mRNAs encode antigens of 7 different SARS-CoV-2 variants. In some embodiments, the mRNAs encode antigens of 8 different SARS-CoV-2 variants. In some embodiments, the mRNAs encode antigens of 9 different SARS-CoV-2 variants. In some embodiments, the mRNAs encode antigens of 10 different SARS-CoV-2 variants. In some embodiments, the mRNAs encode antigens of at least 10 different SARS-CoV-2 variants.

In some embodiments, the mRNAs encode antigens of Alpha variant particles. In some embodiments, the mRNAs encode antigens of Beta variant particles. In some embodiments, the mRNAs encode antigens of Gamma variant particles. In some embodiments, the mRNAs encode antigens of Delta variant particles. In some embodiments, the mRNAs encode antigens of Omicron variant particles. In some embodiments, the mRNAs encode antigens of Alpha, Beta, Gamma, Delta, or Omicron variant particles, or any combination thereof.

In some embodiments, the vaccine comprises intact cells. In some embodiments, the intact cells are proliferation-incompetent. In some embodiments, the cells are rendered proliferation-incompetent by irradiation.

In some embodiments, the vaccine disclosed herein in detail comprises all antigens present in SARS-CoV-2. In some embodiments, the vaccine comprises at least two different SARS-CoV-2 structural protein antigens.

The SARS-CoV-2 antigens disclosed herein can be any SARS-CoV-2 polypeptide or antigen. A skilled artisan would appreciate that SARS-CoV-2 comprises four structural proteins, known as the S (spike), E (envelope), M (membrane), and N (nucleocapsid) proteins; the N protein holds the RNA genome, and the S, E, and M proteins together create the viral envelope. The spike protein comprises the S1 subunit, which catalyzes attachment, the S2 subunit, which is involved in fusion of the virus to the cell and the receptor binding domain (RBD).

In some embodiments, the vaccine comprises at least two different SARS-CoV-2 antigens. In some embodiments, the vaccine comprises two or more different SARS-CoV-2 antigens, including 2, 3, 4, 5, 6, 7, 8, 9, 10 or more antigens. In some embodiments, the vaccine comprises at least 2 different SARS-CoV-2 antigens. In some embodiments, the vaccine comprises 2 different SARS-CoV-2 antigens. In some embodiments, the vaccine comprises 3 different SARS-CoV-2 antigens. In some embodiments, the vaccine comprises 4 different SARS-CoV-2 antigens. In some embodiments, the vaccine comprises 5 different SARS-CoV-2 antigens. In some embodiments, the vaccine comprises 6 different SARS-CoV-2 antigens. In some embodiments, the vaccine comprises 7 different SARS-CoV-2 antigens. In some embodiments, the vaccine comprises 8 different SARS-CoV-2 antigens. In some embodiments, the vaccine comprises 9 different SARS-CoV-2 antigens. In some embodiments, the vaccine comprises 10 different SARS-CoV-2 antigens. In some embodiments, the vaccine comprises at least 10 different SARS-CoV-2 antigens.

In some embodiments, the APCs display at least two different SARS-CoV-2 structural protein antigens. In some embodiments, the APCs display at least two different SARS-CoV-2 antigens comprising antigens of the spike (S) protein, membrane (M) protein, nucleocapsid (N) protein, envelope (E) protein, any part thereof, or any combination thereof. In some embodiments, the APCs display at least two different SARS-CoV-2 antigens comprising antigens of the spike (S) protein or part thereof. In some embodiments, the APCs display at least two different SARS-CoV-2 antigens comprising antigens of the membrane (M) protein or part thereof. In some embodiments, the APCs display at least two different SARS-CoV-2 antigens comprising antigens of the nucleocapsid (N) protein or part thereof. In some embodiments, the APCs display at least two different SARS-CoV-2 antigens comprising antigens of the envelope (E) protein or part thereof. In some embodiments, the APCs display at least two different SARS-CoV-2 antigens comprising antigens of the S1 subunit or part thereof. In some embodiments, the APCs display at least two different SARS-CoV-2 antigens comprising antigens of the S2 subunit or part thereof. In some embodiments, the APCs display at least two different SARS-CoV-2 antigens comprising antigens of the receptor binding domain (RBD) or part thereof.

In some embodiments, the SARS-CoV-2 antigen comprises the S protein, or a part thereof. In some embodiments, the SARS-CoV-2 antigen comprises the S1 subunit. In some embodiments, the SARS-CoV-2 binding antigen comprises the S2 subunit. In some embodiments, the SARS-CoV-2 antigen comprises the receptor binding domain (RBD) or part thereof. In some embodiments, the SARS-CoV-2 antigen comprises the E protein, or a part thereof. In some embodiments, the SARS-CoV-2 antigen comprises the N protein, or a part thereof. In some embodiments, the SARS-CoV-2 antigen comprises the M protein, or a part thereof.

In some embodiments, the vaccine comprises more than one APC population. In some embodiments, the vaccine comprises numerous APC populations, each APC population loaded with a different SARS-CoV-2 variant. In some embodiments, the SARS-CoV-2 variant comprises Alpha, Beta, Gamma, Delta, or Omicron variant particles, or any combination thereof. In some embodiments, the SARS-CoV-2 variant comprises Alpha variant particles. In some embodiments, the SARS-CoV-2 variant comprises Beta variant particles. In some embodiments, the SARS-CoV-2 variant comprises Gamma variant particles. In some embodiments, the SARS-CoV-2 variant comprises Delta variant particles. In some embodiments, the SARS-CoV-2 variant comprises Omicron variant particles.

In some embodiments, the vaccine described herein in detail is produced by a method comprising:
(a) obtaining a population of peripheral blood mononuclear cells (PBMCs) from a subject;
(b) isolating and culturing the APCs from the population of PBMCs;
(c) contacting the APCs with a preparation of SARS-CoV-2 particles for a time period sufficient to generate APCs displaying at least two different SARS-CoV-2 antigens; and
(d) optionally isolating and expanding the APCs from step (c).

In some embodiments, the vaccine described herein in detail is produced by a method comprising:
(e) obtaining a population of peripheral blood mononuclear cells (PBMCs) from a subject;
(f) isolating and culturing the APCs from the population of PBMCs;
(g) contacting the APCs with mRNAs encoding SARS-CoV-2 antigens for a time period sufficient to generate APCs displaying at least two different SARS-CoV-2 antigens; and
(h) optionally isolating and expanding the APCs from step (c).

### Methods for Producing Cell Based Vaccines

In some embodiments, disclosed herein is a method for producing a multi-antigenic autologous, cell-based viral vaccine, comprising autologous antigen presenting cells (APCs) displaying at least two different viral antigens comprising:
a) obtaining a population of peripheral blood mononuclear cells (PBMCs) from a subject;
b) isolating and culturing the APCs from the population of PBMCs;
c) contacting the APCs with a preparation of viral particles for a time period sufficient to generate APCs displaying at least two different viral antigens; and
d) optionally isolating and expanding the APCs from step (c).

In some embodiments, disclosed herein is a method for producing a multi-antigenic autologous, cell-based SARS-CoV-2 vaccine, comprising autologous antigen presenting cells (APCs) displaying at least two different SARS-CoV-2 antigens comprising:
a) obtaining a population of peripheral blood mononuclear cells (PBMCs) from a subject;
b) isolating and culturing the APCs from the population of PBMCs;
c) contacting the APCs with a preparation of SARS-CoV-2 particles for a time period sufficient to generate APCs displaying at least two different SARS-CoV-2 antigens; and
d) optionally isolating and expanding the APCs from step (c).

In some embodiments, disclosed herein is a method for producing a multi-antigenic autologous, cell-based viral vaccine, comprising autologous antigen presenting cells (APCs) displaying at least two different viral antigens comprising:
a) obtaining a population of peripheral blood mononuclear cells (PBMCs) from a subject;
b) isolating and culturing the APCs from the population of PBMCs;
c) contacting the APCs with mRNAs encoding viral antigens for a time period sufficient to generate APCs displaying at least two different viral antigens; and
d) optionally isolating and expanding the APCs from step (c).

In some embodiments, disclosed herein is a method for producing a multi-antigenic autologous, cell-based SARS-CoV-2 vaccine, comprising autologous antigen presenting cells (APCs) displaying at least two different SARS-CoV-2 antigens comprising:
a) obtaining a population of peripheral blood mononuclear cells (PBMCs) from a subject;
b) isolating and culturing the APCs from the population of PBMCs;
c) contacting the APCs with mRNAs encoding SARS-CoV-2 antigens for a time period sufficient to generate APCs displaying at least two different SARS-CoV-2 antigens; and
d) optionally isolating and expanding the APCs from step (c).

In some embodiments, the concentration of APCs isolated from PBMCs is between about 50,000-50×10⁶ cells/ml. In some embodiments, the concentration of APCs isolated from PBMCs is about 50,000 cells/ml. In some embodiments, the concentration of APCs isolated from PBMCs is about 500,000 cells/ml. In some embodiments, the concentration of APCs isolated from PBMCs is about 5×10⁶ cells/ml. In some embodiments, the concentration of APCs isolated from PBMCs is about 50×10⁶ cells/ml.

In some embodiments, any of the APCs disclosed herein in detail are used in the methods disclosed herein. In some embodiments, in order to generate a vaccine with broad specificity and cross-reactivity against more than one SARS-CoV-2 variant, APCs used for the production of the vaccine are loaded with two or more SARS-CoV-2 variants. In some embodiments, APCs loaded with distinct SARS-CoV-2 variants are combined and formulated into one vaccine.

In some embodiments, the preparation of SARS-CoV-2 particles comprises Alpha, Beta, Gamma, Delta, or Omicron variant particles, or any combination thereof. In some embodiments, the preparation of SARS-CoV-2 particles comprises Alpha variant particles. In some embodiments, the preparation of SARS-CoV-2 particles comprises Beta variant particles. In some embodiments, the preparation of SARS-CoV-2 particles comprises Gamma variant particles. In some embodiments, the preparation of SARS-CoV-2 particles comprises Delta variant particles. In some embodiments, the preparation of SARS-CoV-2 particles comprises Omicron variant particles.

A skilled artisan would appreciate that a number of methods for PBMCs isolation exist in the art. In some embodiments, any of them can be applied for producing the APCs disclosed herein.

In some embodiments, PBMCs are separated by density gradient centrifugation. Since each cell has a specific density, density gradient centrifugation can be used for separating the main cell populations, such as lymphocytes, monocytes, granulocytes, and red blood cells, throughout a density gradient medium. In some embodiments, a medium with a density of 1.077 g/ml is used for separating human PBMCs from red blood cells and granulocytes. First, the whole blood is layered over or under a density medium without mixing of the two layers followed, and then the preparation is centrifuged dispersing the cells according to their densities. Following the centrifugation step, the PBMC fraction appears as a thin white layer at the interface between the plasma and the density gradient medium, making it easy to remove the PBMC fraction.

In some embodiments, PBMCs are separated by leukapheresis. A leukapheresis machine is an automated device that separates the inflow of whole blood from the target PBMCs fraction using high-speed centrifugation while returning the outflow material, such as plasma, red blood cells, and granulocytes, back to the donor. The high number of PBMCs obtained in leukapheresis, makes this method ideal for clinical applications. In some embodiments, further processing of the leukaphereis product may be necessary to remove residual red blood cells and granulocytes.

In some embodiments, following separation, PBMCs are further characterized, for example, for quality control purposes. Characterization of PBMCs by flow cytometry allows identification for example of biomarker expression, health of the PBMC, and contaminants, such as red blood cells and granulocytes. In some embodiments, characterization of PBMCs comprises side scatter (SSC) versus forward scatter (FSC), staining for the expression of CD45, and propidium iodide (PI) staining.

A skilled artisan would appreciate that several approaches for APC isolation or enrichment are known from the literature and are used in the clinic. Some of methods for DC isolation are described, for example, in Nair et al. Isolation and Generation of Human Dendritic Cells. Curr Protoc Immunol. 2012 November; 0 7: Unit7.32; which is incorporated herein by reference. Any of these approaches for DC or APC isolation can be applied for producing the vaccines disclosed herein.

In some embodiments, DC isolation comprises:
a. Isolating PBMCs;
b. Depleting monocytes; and
c. Enriching for dendritic cells by metrizamide density gradient centrifugation.

In some embodiments, DC isolation comprises:
a. Isolating PBMCs;
b. Depleting B cells, T cells, and other mononuclear cells; and
c. Enriching for dendritic cells by positive selection using anti-CD83 antibodies.

In some embodiments, the DCs are generated by obtaining monocytes, and then culturing the monocytes with cytokines to induce DCs differentiation.

In some embodiments, the isolated DCs are expanded up to any pre-determined cell number. In some embodiments, the isolated DCs are expanded up to less than about 10×10⁶ cells/ml. In some embodiments, the isolated DCs cells are expanded up to 10×10⁶ cells/ml. In some embodiments, the isolated DCs cells are expanded up to about 25×10⁶ cells/ml. In some embodiments, the isolated DCs cells are expanded up to about 50×10⁶ cells/ml. In some embodiments, the isolated DCs cells are expanded up to about 75×10⁶ cells/ml. In some embodiments, the isolated DCs cells are expanded up to about 100×10⁶ cells/ml. In some embodiments, the isolated DCs cells are expanded up to about 100×10⁶ cells/ml.

In some embodiments, the APCs are contacted with a preparation of SARS-CoV-2 particles at a multiplicity of infection (MOI) of between 0.001 and 10. In some embodiments, the MOI is between 1 to 10, between 0.001 to 0.01, between 0.01 to 0.1, or between 0.1 to 1. In some embodiments the MOI is 1. In some embodiments the MOI is 2. In some embodiments the MOI is 3. In some embodiments the MOI is 4. In some embodiments the MOI is 5. In some embodiments the MOI is 6. In some embodiments the MOI is 7. In some embodiments the MOI is 8. In some embodiments the MOI is 9. In some embodiments the MOI is 10.

An artisan would appreciate that the term “multiplicity of infection” (MOI) may encompass the number of infectious virus particles added per cell.

In some embodiments, the APCs are contacted with a preparation of SARS-CoV-2 particles in the presence of a transduction reagent. In some embodiments, the transduction reagent comprises polybrene (PB). In some embodiments, the transduction reagent comprises Protamine Sulfate.

In some embodiments, the APCs are contacted with a preparation of SARS-CoV-2 particles in medium comprising cAIM media (CTS™ AIM-V, 2% CTS™ GlutaMAX™-I, 1% MEM Sodium Pyruvate, 1% MEM NEAA, 1% MEM vitamins, 0.1% Gentamycin)+50 ng/ml human recombinant GM-CSF+40 ng/mL human recombinant IL-4 (final concentration).

In some embodiments, the APCs are contacted with a preparation of SARS-CoV-2 particles in medium comprising cAIM media+10 ng/mL human recombinant IL-1β+100 ng/mL human recombinant IL-6+25 ng/mL human recombinant TNF-α+1 μg/mL CpG+10 μg/mL PGE2+50 ng/mL LPS (final concentration).

In some embodiments, the APCs are contacted with a preparation of viral particles for a time period sufficient to generate APCs displaying at least two different viral antigens.

In some embodiments, the APCs have been contacted with mRNAs encoding viral antigens for a time period sufficient to generate APCs displaying at least two different viral antigens.

In some embodiments, the time period sufficient to generate APCs displaying at least two different viral antigens is between 1 hour and 180 hours, for example, between 1 hour and 24 hours, between 12 hours and 165 hours, between 24 hours and 180 hours, between 24 hours and 165 hours, between 48 hours and 165 hours, between 60 hours and 180 hours, between 72 hours and 180 hours. In some embodiments, the APCs are contacted for 1 hour. In some embodiments, the APCs are contacted for 2 hours. In some embodiments, the APCs are contacted for 6 hours. In some embodiments, the APCs are contacted for 12 hours. In some embodiments, the APCs are contacted for 24 hours. In some embodiments, the APCs are contacted for 36 hours. In some embodiments, the APCs are contacted for 48 hours. In some embodiments, the APCs are contacted for 60 hours. In some embodiments, the APCs are contacted for 72 hours. In some embodiments, the APCs are contacted for 165 hours. In some embodiments, the APCs are contacted for 180 hours.

The incubation temperature may range from about 4° C. to about 37° C., from about 25° C. to about 37° C., about 4° C., about 25° C., or about 37° C. In some embodiments, the incubation temperature is 37° C.

In some embodiments, the preparation of viral particles comprises Severe acute respiratory syndrome coronavirus 2 (SARS-CoV-2), SARS-CoV-1, Middle East respiratory syndrome (MERS), MERS-CoV-1, Adenovirus (ADV), Herpes simplex virus (HSV), Herpes simplex-type 1, Herpes simplex-type 2, Human herpesvirus-type 8, Epstein-Barr virus (EBV), Human cytomegalovirus (CMV), varicella zoster virus (VZV), Human papillomavirus (HPV), Bocavirus (BoV), Hepatitis C Virus (HCV), yellow fever virus, dengue virus, Zika virus, West Nile virus, Japanese encephalitis virus (JEV), polio, Rhinovirus, Ebola virus, Marburg virus, Influenzavirus, Influenzavirus A, Influenzavirus B, Influenzavirus C, Influenzavirus D, Thogotovirus, Quaranjavirus, Measles virus, Parainfluenza virus, Respiratory syncytial virus (RSV), Metapneumovirus (MPV), Rabies virus, Human T-cell lymphotropic virus type 1 (HTLV-1), human immunodeficiency virus (HIV), chikungunya virus, or Hepatitis B Virus (HBV) viral particles.

In some embodiments, the preparation of viral particles comprises SARS-CoV-2 particles. In some embodiments, the preparation of viral particles comprises SARS-CoV-1 particles. In some embodiments, the preparation of viral particles comprises MERS particles. In some embodiments, the preparation of viral particles comprises MERS-CoV-1 particles. In some embodiments, the preparation of viral particles comprises ADV particles. In some embodiments, the preparation of viral particles comprises Herpes simplex virus (HSV) particles. In some embodiments, the preparation of viral particles comprises Herpes simplex-type 1 particles. In some embodiments, the preparation of viral particles comprises Herpes simplex-type 2 particles. In some embodiments, the preparation of viral particles comprises Herpes virus-type 8 particles. In some embodiments, the preparation of viral particles comprises EBV particles. In some embodiments, the preparation of viral particles comprises CMV particles. In some embodiments, the preparation of viral particles comprises VZV particles. In some embodiments, the preparation of viral particles comprises HPV particles. In some embodiments, the preparation of viral particles comprises BoV particles. In some embodiments, the preparation of viral particles comprises HCV particles. In some embodiments, the preparation of viral particles comprises yellow fever virus particles. In some embodiments, the preparation of viral particles comprises dengue virus particles. In some embodiments, the preparation of viral particles comprises Zika virus particles. In some embodiments, the preparation of viral particles comprises West Nile virus. In some embodiments, the preparation of viral particles comprises Japanese encephalitis virus particles. In some embodiments, the preparation of viral particles comprises polio virus particles. In some embodiments, the preparation of viral particles comprises Rhinovirus particles. In some embodiments, the preparation of viral particles comprises Ebola virus particles. In some embodiments, the preparation of viral particles comprises Marburg virus. In some embodiments, the preparation of viral particles comprises Influenzavirus particles. In some embodiments, the preparation of viral particles comprises Influenzavirus A particles. In some embodiments, the preparation of viral particles comprises Influenzavirus B particles. In some embodiments, the preparation of viral particles comprises Influenzavirus C particles. In some embodiments, the preparation of viral particles comprises Influenzavirus D particles. In some embodiments, the preparation of viral particles comprises Thogotovirus particles. In some embodiments, the preparation of viral particles comprises Quaranjavirus particles. In some embodiments, the preparation of viral particles comprises Measles virus particles. In some embodiments, the preparation of viral particles comprises Parainfluenza virus particles. In some embodiments, the preparation of viral particles comprises RSV particles. In some embodiments, the preparation of viral particles comprises MPV particles. In some embodiments, the preparation of viral particles comprises rabies virus particles. In some embodiments, the preparation of viral particles comprises HTLV-1 particles. In some embodiments, the preparation of viral particles comprises HIV particles. In some embodiments, the preparation of viral particles comprises chikungunya viral particles. In some embodiments, the preparation of viral particles comprises HBV particles.

In some embodiments, the preparation of viral particles comprises viruses in the Baltimore classification Group I group of viruses of double-stranded DNA viruses (e.g. Adenoviruses, Herpesviruses including Epstein-Barr virus, Poxviruses, Polyoma viruses including BK virus and JC virus (human polyomavirus 2)). In some embodiments, the preparation of viral particles comprises viruses in the Baltimore classification Group II group of viruses of single-stranded (or “sense”) DNA viruses (e.g. Parvoviruses). In some embodiments, the preparation of viral particles comprises viruses in the Baltimore classification Group III group of viruses of double-stranded RNA viruses (e.g. Reoviruses). In some embodiments, the preparation of viral particles comprises viruses in the Baltimore classification Group IV group of viruses of single-stranded (sense) RNA viruses (e.g. Picornaviruses, Togaviruses, Coronavirus including SARS-CoV-2). In some embodiments, the preparation of viral particles comprises viruses in the Baltimore classification Group V of viruses of single-stranded (antisense) RNA viruses (e.g. Orthomyxoviruses, Rhabdoviruses). In some embodiments, the preparation of viral particles comprises viruses in the Baltimore classification Group VI group of viruses of single-stranded (sense) RNA viruses with DNA intermediate in life-cycle (e.g. Retroviruses). In some embodiments, the preparation of viral particles comprises viruses in the Baltimore classification Group VII group of viruses of double-stranded DNA viruses with RNA intermediate in life-cycle (e.g. Hepadnaviruses).

In some embodiments, the APCs have been contacted with mRNAs encoding viral antigens for a time period sufficient to generate APCs displaying at least two different viral antigens.

In some embodiments, the mRNAs encode viral antigens comprising Severe acute respiratory syndrome coronavirus 2 (SARS-CoV-2), SARS-CoV-1, Middle East respiratory syndrome (MERS), MERS-CoV-1, Adenovirus (ADV), Herpes simplex virus (HSV), Herpes simplex-type 1, Herpes simplex-type 2, Human herpesvirus-type 8, Epstein-Barr virus (EBV), Human cytomegalovirus (CMV), varicella zoster virus (VZV), Human papillomavirus (HPV), Bocavirus (BoV), Hepatitis C Virus (HCV), yellow fever virus, dengue virus, Zika virus, West Nile virus, Japanese encephalitis virus (JEV), polio, Rhinovirus, Ebola virus, Marburg virus, Influenzavirus, Influenzavirus A, Influenzavirus B, Influenzavirus C, Influenzavirus D, Thogotovirus, Quaranjavirus, Measles virus, Parainfluenza virus, Respiratory syncytial virus (RSV), Metapneumovirus (MPV), Rabies virus, Human T-cell lymphotropic virus type 1 (HTLV-1), human immunodeficiency virus (HIV), chikungunya virus, or Hepatitis B Virus (HBV) antigens.

In some embodiments, the mRNAs encode viral antigens comprising SARS-CoV-2 antigens. In some embodiments, the mRNAs encode viral antigens comprisingSARS-CoV-1 particles. In some embodiments, the mRNAs encode viral antigens comprising MERS antigens. In some embodiments, the mRNAs encode viral antigens comprising MERS-CoV-1 antigens. In some embodiments, the mRNAs encode viral antigens comprising ADV antigens. In some embodiments, the mRNAs encode viral antigens comprising Herpes simplex virus (HSV) antigens. In some embodiments, the mRNAs encode viral antigens comprising Herpes simplex-type 1 antigens. In some embodiments, the mRNAs encode viral antigens comprising Herpes simplex-type 2 antigens. In some embodiments, the mRNAs encode viral antigens comprising Herpes virus-type 8 antigens. In some embodiments, the mRNAs encode viral antigens comprising EBV antigens. In some embodiments, the mRNAs encode viral antigens comprising CMV antigens. In some embodiments, the mRNAs encode viral antigens comprising VZV antigens. In some embodiments, the mRNAs encode viral antigens comprising HPV antigens. In some embodiments, the mRNAs encode viral antigens comprising BoV antigens. In some embodiments, the mRNAs encode viral antigens comprising HCV antigens. In some embodiments, the mRNAs encode viral antigens comprising yellow fever virus antigens. In some embodiments, the mRNAs encode viral antigens comprising dengue virus antigens. In some embodiments, the mRNAs encode viral antigens comprising Zika virus antigens. In some embodiments, the mRNAs encode viral antigens comprising West Nile virus. In some embodiments, the mRNAs encode viral antigens comprising Japanese encephalitis virus antigens. In some embodiments, the mRNAs encode viral antigens comprising polio virus antigens. In some embodiments, the mRNAs encode viral antigens comprising Rhinovirus antigens. In some embodiments, the mRNAs encode viral antigens comprising Ebola virus antigens. In some embodiments, the mRNAs encode viral antigens comprising Marburg virus. In some embodiments, the mRNAs encode viral antigens comprising Influenzavirus antigens. In some embodiments, the mRNAs encode viral antigens comprising Influenzavirus A antigens. In some embodiments, the mRNAs encode viral antigens comprising Influenzavirus B antigens. In some embodiments, the mRNAs encode viral antigens comprising Influenzavirus C antigens. In some embodiments, the mRNAs encode viral antigens comprising Influenzavirus D antigens. In some embodiments, the mRNAs encode viral antigens comprising Thogotovirus antigens. In some embodiments, the mRNAs encode viral antigens comprising Quaranja virus antigens. In some embodiments, the mRNAs encode viral antigens comprising Measles virus antigens. In some embodiments, the mRNAs encode viral antigens comprising Parainfluenza virus antigens. In some embodiments, the mRNAs encode viral antigens comprising RSV antigens. In some embodiments, the mRNAs encode viral antigens comprising MPV antigens. In some embodiments, the mRNAs encode viral antigens comprising rabies virus antigens. In some embodiments, the mRNAs encode viral antigens comprising HTLV-1 antigens. In some embodiments, the mRNAs encode viral antigens comprising HIV antigens. In some embodiments, the mRNAs encode viral antigens comprising chikungunya viral antigens. In some embodiments, the mRNAs encode viral antigens comprising HBV antigens.

In some embodiments, the mRNAs encode viral antigens of viruses in the Baltimore classification Group I group of viruses of double-stranded DNA viruses (e.g. Adenoviruses, Herpesviruses including Epstein-Barr virus, Poxviruses, Polyoma viruses including BK virus and JC virus (human polyomavirus 2)). In some embodiments, the mRNAs encode viral antigens of viruses in the viruses in the Baltimore classification Group II group of viruses of single-stranded (or “sense”) DNA viruses (e.g. Parvoviruses). In some embodiments, the mRNAs encode viral antigens of viruses in the Baltimore classification Group III group of viruses of double-stranded RNA viruses (e.g. Reoviruses). In some embodiments, the mRNAs encode viral antigens of viruses in the Baltimore classification Group IV group of viruses of single-stranded (sense) RNA viruses (e.g. Picornaviruses, Togaviruses, Coronavirus including SARS-CoV-2). In some embodiments, the mRNAs encode viral antigens of viruses in the Baltimore classification Group V of viruses of single-stranded (antisense) RNA viruses (e.g. Orthomyxoviruses, Rhabdoviruses). In some embodiments, the mRNAs encode viral antigens of viruses in the Baltimore classification Group VI group of viruses of single-stranded (sense) RNA viruses with DNA intermediate in life-cycle (e.g. Retroviruses). In some embodiments, the mRNAs encode viral antigens of viruses in the Baltimore classification Group VII group of viruses of double-stranded DNA viruses with RNA intermediate in life-cycle (e.g. Hepadnaviruses).

In some embodiments, the mRNAs encode structural protein antigens. In some embodiments, the mRNAs encode viral antigens comprising antigens of the spike (S) protein, membrane (M) protein, nucleocapsid (N) protein, envelope (E) protein, glycoprotein (G), any part thereof, or any combination thereof. In some embodiments, the mRNAs encode viral antigens comprising antigens of the spike (S) protein or part thereof. In some embodiments, the mRNAs encode viral antigens comprising antigens of the membrane (M) protein or part thereof. In some embodiments, the mRNAs encode viral antigens comprising antigens of the nucleocapsid (N) protein or part thereof. In some embodiments, the mRNAs encode viral antigens comprising antigens of the envelope (E) protein or part thereof. In some embodiments, the mRNAs encode viral antigens comprising antigens of the glycoprotein (G) or part thereof. In some embodiments, the mRNAs encode viral antigens comprising antigens of the receptor binding domain (RBD) or part thereof.

In some embodiments, the APCs (i.e., DCs) can be isolated (or purified) prior to administration to the subject. Purification of the cells can be done using a variety of methods known in the art, including methods in which antibodies to specific cell surface molecules are employed. These methods include both positive and negative selection methods. For example, cells generated in vitro can be isolated by staining the cells with fluorescently labeled antibodies to cell surface markers followed by sorting of the cells that express these markers on their cell surface using fluorescence activated cell sorting (FACS). These and other purification/isolation methods are well known to those of skill in the art.

In some embodiments, T cells are isolated from PBMCs. In some embodiments, the T cells are used for ex-vivo quality control (QC) tests. In some embodiments, the APCs loaded with the antigen can be used to stimulate CTL proliferation in vivo or ex vivo. The ability of the loaded APCs to stimulate a CTL response can be measured by assaying the ability of the effector cells to lyse target cells.

A variety of in vitro and in vivo assays are known in the art for measuring an immune response, including measuring humoral and cellular immune responses, which include but are not limited to, standard immunoassays, such as RIA, ELISA assays; intracellular staining; T cell assays including for example, lymphoproliferation (lymphocyte activation) assays, CTL cytotoxic cell assays, or by assaying for T-lymphocytes specific for the antigen in a sensitized subject. Such assays are well known in the art. In some embodiments, the release of cytokines (e.g., IFN-γ, TNF-α, and/or IL-17) may be assayed by, e.g., ELISpot assay, to determine immune responses.

In some embodiments, the method further comprises the step of transducing the APCs with a vector encoding a cytokine. In some embodiments, the cytokine is selected from the group consisting of granulocyte macrophage colony-stimulating factor (GM-CSF), interferon alpha (IFN-α), interleukin-2 (IL-2), interleukin-12 (IL-12), tumor necrosis factor alpha (TNF-α), and any combination thereof. In some embodiments, the APCs express GM-CSF. In some embodiments, the APCs express IFN-α. In some embodiments, the APCs express IL-2. In some embodiments, the APCs express IL-12. In some embodiments, the APCs express TNF-α.

In some embodiments, the method further comprises the step of transducing the APCs with a vector encoding CD40L, CD80, 4-1BBL, CD40, and MBL2, or any combination thereof. In some embodiments, the APCs express CD40L. In some embodiments, the APCs express CD80. In some embodiments, the APCs express 4-1BBL. In some embodiments, the APCs express CD40. In some embodiments, the APCs express MBL2. In some embodiments, the cytokine, CD40L, CD80, 4-1BBL, CD40, or MBL2 are encoded by a vector transduced to the APCs. In some embodiments, the vector comprises a viral vector or a non-viral vector. In some embodiments, the vector comprises a viral vector. In some embodiments, the vector comprises a non-viral vector.

A skilled artisan would appreciate that several methods are well known in the art for transducing mammalian cells. Any of these methods can be applied for producing the vaccines disclosed herein. Transduction methods are disclosed, for example in O'Keefe E. Nucleic Acid Delivery: Lentiviral and Retroviral Vectors. Mater Methods. 2013; 3:174; and in Gouvarchin Ghaleh H E et al. Concise review on optimized methods in production and transduction of lentiviral vectors in order to facilitate immunotherapy and gene therapy. Biomedicine & Pharmacotherapy. Vol 128, August 2020; both of which are incorporated herein by reference.

### Pharmaceutical Compositions

In some embodiments, disclosed herein is a pharmaceutical composition comprising the vaccine described herein in detail. In some embodiments, the pharmaceutical composition comprises the APCs described herein in detail. In some embodiments, the pharmaceutical composition comprises APCs displaying at least two different viral antigens. In some embodiments, the present pharmaceutical composition can be used for prevention and/or treatment of viral infections or a viral disease.

In some embodiments, the pharmaceutical composition comprises APCs displaying at least two different SARS-CoV-2 antigens. In some embodiments, the present pharmaceutical composition can be used for prevention and/or treatment of SARS-CoV-2 infection or COVID-19.

In some embodiments, the present pharmaceutical composition comprises APCs contacted in vitro or ex vivo with at least SARS-CoV-2 antigens. In some embodiments, the present pharmaceutical composition comprises APCs contacted in vitro with nucleic acids encoding at least one SARS-CoV-2 antigen.

In some embodiments, the present pharmaceutical composition can be useful as a vaccine for prophylactic or therapeutic treatment of a viral infection in a subject. In some embodiments, the present pharmaceutical composition can be useful as a vaccine for prophylactic or therapeutic treatment of a SARS-CoV-2 infection in a subject.

Administration of the vaccines described herein, in pure form or in an appropriate pharmaceutical composition, can be carried out via any of the accepted modes of administration of agents for serving similar utilities. The pharmaceutical compositions comprising autologous APCs, and optionally other relevant pharmaceutically active ingredients, can be prepared by formulating autologous APCs with an appropriate physiologically acceptable carrier, diluent or excipient; and may be formulated into preparations in liquid forms, such as solutions, injections, and inhalants. In addition, suitable excipients such as salts, buffers and stabilizers may be present within the composition.

Administration may be achieved by different routes, including oral, parenteral, nasal, intravenous, or topical. An amount that, following administration, reduces, inhibits, prevents or delays the progression of the virus is considered effective. A skilled artisan would appreciate that the term “physiologically acceptable carrier, diluent or excipient”, may in some embodiments be used interchangeably with the term “pharmaceutically acceptable carrier” having all the same means and qualities.

A skilled artisan would appreciate that the term “treating” and grammatical forms thereof, may in some embodiments encompass both therapeutic treatment and prophylactic or preventative measures with respect to a viral disease, such as COVID-19, as described herein, wherein the objective is to prevent or lessen a viral infection, including a SARS-CoV-2 infection, as described herein. Thus, in some embodiments of methods disclosed herein, treating may include directly affecting or curing, suppressing, inhibiting, preventing, reducing the severity of, delaying the onset of, reducing symptoms associated with a viral disease, such as COVID-19. Thus, in some embodiments, “treating” encompasses preventing, delaying progression, inhibiting the growth of, delaying disease progression, reducing viral load, reducing the incidence of, expediting remission, inducing remission, augmenting remission, speeding recovery, increasing efficacy of or decreasing resistance to alternative therapeutics, or a combination thereof. In some embodiments, “preventing” encompasses delaying the onset of symptoms, preventing relapse to a disease, decreasing the number or frequency of relapse episodes, increasing latency between symptomatic episodes, or a combination thereof. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, a viral disease or viral infection is caused by SARS-CoV-2. In some embodiments, a viral disease or viral infection is caused by SARS-CoV-1. In some embodiments, a viral disease or viral infection is caused by MERS. In some embodiments, a viral disease or viral infection is caused by MERS-CoV-1. In some embodiments, a viral disease or viral infection is caused by ADV. In some embodiments, a viral disease or viral infection is caused by Herpes simplex virus (HSV). In some embodiments, a viral disease or viral infection is caused by Herpes simplex-type 1. In some embodiments, a viral disease or viral infection is caused by Herpes simplex-type 2. In some embodiments, a viral disease or viral infection is caused by Herpes virus-type 8. In some embodiments, a viral disease or viral infection is caused by EBV. In some embodiments, a viral disease or viral infection is caused by CMV. In some embodiments, a viral disease or viral infection is caused by VZV. In some embodiments, a viral disease or viral infection is caused by HPV. In some embodiments, a viral disease or viral infection is caused by BoV. In some embodiments, a viral disease or viral infection is caused by HCV. In some embodiments, a viral disease or viral infection is caused by yellow fever virus. In some embodiments, a viral disease or viral infection is caused by dengue virus. In some embodiments, a viral disease or viral infection is caused by Zika virus. In some embodiments, a viral disease or viral infection is caused by West Nile virus. In some embodiments, a viral disease or viral infection is caused by Japanese encephalitis virus. In some embodiments, a viral disease or viral infection is caused by polio virus. In some embodiments, a viral disease or viral infection is caused by Rhinovirus. In some embodiments, a viral disease or viral infection is caused by Ebola virus. In some embodiments, a viral disease or viral infection is caused by Marburg virus. In some embodiments, a viral disease or viral infection is caused by Influenzavirus. In some embodiments, a viral disease or viral infection is caused by Influenzavirus A. In some embodiments, a viral disease or viral infection is caused by Influenzavirus B. In some embodiments, a viral disease or viral infection is caused by Influenzavirus C. In some embodiments, a viral disease or viral infection is caused by Influenzavirus D. In some embodiments, a viral disease or viral infection is caused by Thogotovirus. In some embodiments, a viral disease or viral infection is caused by Quaranjavirus. In some embodiments, a viral disease or viral infection is caused by Measles virus. In some embodiments, a viral disease or viral infection is caused by Parainfluenza virus. In some embodiments, a viral disease or viral infection is caused by RSV. In some embodiments, a viral disease or viral infection is caused by MPV. In some embodiments, a viral disease or viral infection is caused by rabies virus. In some embodiments, a viral disease or viral infection is caused by HTLV-1. In some embodiments, a viral disease or viral infection is caused by HIV. In some embodiments, a viral disease or viral infection is caused by chikungunya virus. In some embodiments, a viral disease or viral infection is caused by HBV.

In some embodiments, the viral disease or viral infection is caused by viruses in the Baltimore classification Group I group of viruses of double-stranded DNA viruses (e.g. Adenoviruses, Herpesviruses including Epstein-Barr virus, Poxviruses, Polyoma viruses including BK virus and JC virus (human polyomavirus 2)). In some embodiments, the viral disease or viral infection is caused by viruses in the Baltimore classification Group II group of viruses of single-stranded (or “sense”) DNA viruses (e.g. Parvoviruses). In some embodiments, the viral disease or viral infection is caused by viruses in the Baltimore classification Group III group of viruses of double-stranded RNA viruses (e.g. Reoviruses). In some embodiments, the viral disease or viral infection is caused by viruses in the Baltimore classification Group IV group of viruses of single-stranded (sense) RNA viruses (e.g. Picornaviruses, Togaviruses, Coronavirus including SARS-CoV-2). In some embodiments, the viral disease or viral infection is caused by viruses in the Baltimore classification Group V of viruses of single-stranded (antisense) RNA viruses (e.g. Orthomyxoviruses, Rhabdoviruses). In some embodiments, the viral disease or viral infection is caused by viruses in the Baltimore classification Group VI group of viruses of single-stranded (sense) RNA viruses with DNA intermediate in life-cycle (e.g. Retroviruses). In some embodiments, the viral disease or viral infection is caused by viruses in the Baltimore classification Group VII group of viruses of double-stranded DNA viruses with RNA intermediate in life-cycle (e.g. Hepadnaviruses).

In some embodiments, a symptom comprises pneumonia, acute respiratory distress syndrome (ARDS), multi-organ failure, fever, dry cough, fatigue, sputum production, loss of smell, loss of appetite, shortness of breath, muscle pain, joint pain, chest pain, sore throat, headache, chills, nausea, vomiting, nasal congestion, runny nose, diarrhea, or a combination thereof. In one embodiment, the symptom comprises pneumonia. In another embodiment, the symptom comprises ARDS. In another embodiment, the symptom comprises multi-organ failure. In another embodiment, the symptom comprises a fever. In another embodiment, the symptom comprises a dry cough. In another embodiment, the symptom comprises fatigue. In another embodiment, the symptom comprises sputum production. In another embodiment, the symptom comprises loss of smell. In another embodiment, the symptom comprises loss of appetite. In another embodiment, the symptom comprises shortness of breath. In another embodiment, the symptom comprises muscle pain. In another embodiment, the symptom comprises joint pain. In another embodiment, the symptom comprises chest pain. In another embodiment, the symptom comprises a sore throat. In another embodiment, the symptom comprises a headache. In another embodiment, the symptom comprises chills. In another embodiment, the symptom comprises nausea. In another embodiment, the symptom comprises vomiting. In another embodiment, the symptom comprises nasal congestion. In another embodiment, the symptom comprises a runny nose. In another embodiment, the symptom comprises diarrhea. In another embodiment, the symptom comprises elevated cytokine levels. In another embodiment, the symptom comprises elevated IL-6 levels. In another embodiment, the symptom comprises elevated IL-8 levels. In another embodiment, the symptom comprises elevated IL-17A levels.

In some embodiments, the subject is a human subject. In some embodiments, the subject is a human child. In some embodiments, the subject is an adult human. In some embodiments, the subject is a non-human mammal.

In some embodiments, the amount of APCs administered is sufficient to result in a reduction in viral load. In some embodiments, the amount of APCs administered is sufficient to result in clinically relevant reduction in disease symptoms as would be known to the skilled clinician.

The precise dosage and duration of treatment is a function of the subject conditions and may be determined empirically using known testing protocols or by testing the compositions in model systems known in the art and extrapolating therefrom. Controlled clinical trials may also be performed. Dosages may also vary with the COVID-19 severity of the subject to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The APCs vaccine may be administered alone or in combination with other known anti-viral treatments. In some embodiments, the vaccine is administered together with remdesivir. In some embodiments, the vaccine is administered together with over the counter cold medications. In some embodiments, the vaccine is administered together with oxygen therapy. In some embodiments, the vaccine is administered together with intravenous fluids. In some embodiments, the vaccine is administered together with convalescent plasma. In some embodiments, the vaccine is administered together with dexamethasone. In some embodiments, the vaccine is administered together with steroids. In some embodiments, the vaccine is administered together with systemic corticosteroids.

In some embodiments, the vaccine is administered together with ACE-inhibitors. In some embodiments, the vaccine is administered together with angiotensin receptor blockers. In some embodiments, the vaccine is administered together with vitamin D. In some embodiments, the vaccine is administered together with baloxavir marboxil. In some embodiments, the vaccine is administered together with favipiravir. In some embodiments, the vaccine is administered together with lopinavir/ritonavir. In some embodiments, the vaccine is administered together with ruxolitinib.

In some embodiments, the vaccine is administered together with chloroquine. In some embodiments, the vaccine is administered together with hydroxychloroquine. In some embodiments, the vaccine is administered together with interferon beta 1. In some embodiments, the vaccine is administered together with cholchicine. In some embodiments, the vaccine is administered together with baricitinib.

Typical routes of administering the vaccine and/or other anti-viral agents administered with them include, without limitation, systemic, intravenous, oral, topical, inhalation, parenteral, sublingual, buccal, and intranasal. The term parenteral as used herein includes subcutaneous injections, intravenous, intramuscular, intrasternal injection or infusion techniques. Pharmaceutical compositions according to some embodiments as described herein, are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described anti-viral agent may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of APCs of the present disclosure, for treatment of a disease or condition of interest in accordance with teachings herein.

A pharmaceutical composition may be in the form of a solid or liquid. In one embodiment, the pharmaceutically acceptable carrier(s) are particulate, so that the compositions are, for example, in tablet or powder form. The pharmaceutically acceptable carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of the anti-viral agents herein disclosed such that a suitable dosage will be obtained. When intended for oral administration, this amount may be varied to be between 0.1 and about 70% of the weight of the composition. Certain oral pharmaceutical compositions contain between about 4% and about 75% of the anti-viral agents. In some embodiments, pharmaceutical compositions and preparations according to the embodiments described herein, are prepared so that a parenteral dosage unit contains between 0.01 to 10% by weight of the anti-viral agents.

The pharmaceutical composition may be intended for topical administration, in which case the pharmaceutically acceptable carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration.

The pharmaceutical compositions may be prepared by methodology well known in the pharmaceutical art. For example, a pharmaceutical composition intended to be administered by injection can be prepared by combining a composition that comprises the APCs and/or other anti-viral agents as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the active ingredients so as to facilitate dissolution or homogeneous suspension of the active ingredients in the aqueous delivery system.

The compositions may be administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the COVID-19; and the subject undergoing therapy.

Compositions comprising the APCs described herein may also be administered simultaneously with, prior to, or after administration of one or more other therapeutic agents. Such combination therapy may include administration of a single pharmaceutical dosage formulation which contains a compound as disclosed herein, and one or more additional active agents, as well as administration of compositions comprising the APCs disclosed herein, and each active agent in its own separate pharmaceutical dosage formulation. For example, the APCs as described herein, and the other active agent can be administered to the patient together in a single parenteral dosage composition such as in a saline solution or other physiologically acceptable solution, or each agent administered in separate parenteral dosage formulations. Where separate dosage formulations are used, the compositions comprising the APCs, and one or more additional active agents can be administered at essentially the same time, i.e., concurrently, or at separately staggered times, i.e., sequentially and in any order; combination therapy is understood to include all these regimens.

The compositions comprising the herein described vaccines may be administered to an individual afflicted with a viral disease, such as COVID-19. In some embodiments, the vaccine described herein is incorporated into a pharmaceutical composition prior to administration. A pharmaceutical composition comprises one or more of the APCs described herein in combination with a pharmaceutically acceptable carrier or excipient as described elsewhere herein. To prepare a pharmaceutical composition, an effective amount of the APCs is mixed with any pharmaceutically acceptable carrier(s) or excipient known to those skilled in the art to be suitable for the particular mode of administration.

A pharmaceutically acceptable carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens, phenols or cresols, mercurials, chlorobutanol, methyl and propyl p-hydroxybenzoic acid esters, thimerosal, benzalkonium chloride and benzethonium chloride); antioxidants (such as ascorbic acid and sodium bisulfite; methionine, sodium thiosulfate, platinum, catalase, citric acid, cysteine, thioglycerol, thioglycolic acid, thiosorbitol, butylated hydroxyanisol, butylated hydroxytoluene, and/or propyl gallate) and chelating agents (such as ethylenediaminetetraacetic acid (EDTA)); buffers (such as acetates, citrates and phosphates). If administered intravenously, suitable pharmaceutically acceptable carriers include physiological saline or phosphate buffered saline (PBS), and solutions containing thickening and solubilizing agents, such as glucose, polyethylene glycol, polypropylene glycol and mixtures thereof.

For any preparation used in the methods disclosed herein, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p.1].

Depending on the severity and responsiveness of the condition to be treated, dosing can be of a single or a plurality of administrations, with course of treatment lasting from several days to several weeks or until cure is effected or diminution of the disease state is achieved. The amount of a composition to be administered will, of course, be dependent on e.g. the subject being treated, the severity of the affliction, the manner of administration, the judgment of the prescribing physician, etc.

Compositions of some embodiments, may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation formulated in a compatible pharmaceutical carrier may also be prepared, placed in an appropriate container, and labeled for treatment of an indicated condition, as is further detailed above.

### Methods of Preventing or Treating Viral Infection

According to the methods of the present disclosure, vaccines described herein may be used for the prevention and/or treatment of viral infection or viral disease in a subject, either alone or in combination with other methods suitable for the prevention and/or treatment of viral infections.

In some embodiments, disclosed herein is a method for preventing or treating a viral infection in a subject comprising:
a) obtaining a population of peripheral blood mononuclear cells (PBMCs) from a subject;
b) isolating and culturing the APCs from the population of PBMCs;
c) contacting the APCs with a preparation of viral particles for a time period sufficient to generate APCs displaying at least two different virus antigens;
d) optionally isolating and expanding the APCs from step (c); and
e) administering the APCs to the subject.

In some embodiments, disclosed herein is a method for preventing or treating a viral infection in a subject comprising:
a) obtaining a population of peripheral blood mononuclear cells (PBMCs) from a subject;
b) isolating and culturing the APCs from the population of PBMCs;
c) contacting the APCs with mRNAs encoding viral antigens for a time period sufficient to generate APCs displaying at least two different virus antigens;
d) optionally isolating and expanding the APCs from step (c); and
e) administering the APCs to the subject.

In some embodiments, the present disclosure provides a method of preventing and/or treating a viral infection in a subject, the method comprising the step of administering to the subject the vaccine described herein in detail.

In some embodiments, the present disclosure provides a method of eliciting an immune response to virus in a subject, the method comprising the step of administering to the subject the vaccine described herein in detail.

In some embodiments, a viral infection comprises a SARS-CoV-2, SARS-CoV-1, Middle East respiratory syndrome (MERS), MERS-CoV-1, Adenovirus (ADV), Herpes simplex virus (HSV), Herpes simplex-type 1, Herpes simplex-type 2, Human herpesvirus-type 8, Epstein-Barr virus (EBV), Human cytomegalovirus (CMV), varicella zoster virus (VZV), Human papillomavirus (HPV), Bocavirus (BoV), Hepatitis C Virus (HCV), yellow fever virus, dengue virus, Zika virus, West Nile virus, Japanese encephalitis virus (JEV), polio, Rhinovirus, Ebola virus, Marburg virus, Influenzavirus, Influenzavirus A, Influenzavirus B, Influenzavirus C, Influenzavirus D, Thogotovirus, Quaranjavirus, Measles virus, Parainfluenza virus, Respiratory syncytial virus (RSV), Metapneumovirus (MPV), Rabies virus, Human T-cell lymphotropic virus type 1 (HTLV-1), human immunodeficiency virus (HIV), chikungunya virus, or Hepatitis B Virus (HBV) infection.

In some embodiments, a viral disease or viral infection is caused by SARS-CoV-2. In some embodiments, a viral disease or viral infection is caused by SARS-CoV-1. In some embodiments, a viral disease or viral infection is caused by MERS. In some embodiments, a viral disease or viral infection is caused by MERS-CoV-1. In some embodiments, a viral disease or viral infection is caused by ADV. In some embodiments, a viral disease or viral infection is caused by Herpes simplex virus (HSV). In some embodiments, a viral disease or viral infection is caused by Herpes simplex-type 1. In some embodiments, a viral disease or viral infection is caused by Herpes simplex-type 2. In some embodiments, a viral disease or viral infection is caused by Herpes virus-type 8. In some embodiments, a viral disease or viral infection is caused by EBV. In some embodiments, a viral disease or viral infection is caused by CMV. In some embodiments, a viral disease or viral infection is caused by VZV. In some embodiments, a viral disease or viral infection is caused by HPV. In some embodiments, a viral disease or viral infection is caused by BoV. In some embodiments, a viral disease or viral infection is caused by HCV. In some embodiments, a viral disease or viral infection is caused by yellow fever virus. In some embodiments, a viral disease or viral infection is caused by dengue virus. In some embodiments, a viral disease or viral infection is caused by Zika virus. In some embodiments, a viral disease or viral infection is caused by West Nile virus. In some embodiments, a viral disease or viral infection is caused by Japanese encephalitis virus. In some embodiments, a viral disease or viral infection is caused by polio virus. In some embodiments, a viral disease or viral infection is caused by Rhinovirus. In some embodiments, a viral disease or viral infection is caused by Ebola virus. In some embodiments, a viral disease or viral infection is caused by Marburg virus. In some embodiments, a viral disease or viral infection is caused by Influenzavirus. In some embodiments, a viral disease or viral infection is caused by Influenzavirus A. In some embodiments, a viral disease or viral infection is caused by Influenzavirus B. In some embodiments, a viral disease or viral infection is caused by Influenzavirus C. In some embodiments, a viral disease or viral infection is caused by Influenzavirus D. In some embodiments, a viral disease or viral infection is caused by Thogotovirus. In some embodiments, a viral disease or viral infection is caused by Quaranjavirus. In some embodiments, a viral disease or viral infection is caused by Measles virus. In some embodiments, a viral disease or viral infection is caused by Parainfluenza virus. In some embodiments, a viral disease or viral infection is caused by RSV. In some embodiments, a viral disease or viral infection is caused by MPV. In some embodiments, a viral disease or viral infection is caused by rabies virus. In some embodiments, a viral disease or viral infection is caused by HTLV-1. In some embodiments, a viral disease or viral infection is caused by HIV. In some embodiments, a viral disease or viral infection is caused by chikungunya virus. In some embodiments, a viral disease or viral infection is caused by HBV.

In some embodiments, the viral disease or viral infection is caused by viruses in the Baltimore classification Group I group of viruses of double-stranded DNA viruses (e.g. Adenoviruses, Herpesviruses including Epstein-Barr virus, Poxviruses, Polyoma viruses including BK virus and JC virus (human polyomavirus 2)). In some embodiments, the viral disease or viral infection is caused by viruses in the Baltimore classification Group II group of viruses of single-stranded (or “sense”) DNA viruses (e.g. Parvoviruses). In some embodiments, the viral disease or viral infection is caused by viruses in the Baltimore classification Group III group of viruses of double-stranded RNA viruses (e.g. Reoviruses). In some embodiments, the viral disease or viral infection is caused by viruses in the Baltimore classification Group IV group of viruses of single-stranded (sense) RNA viruses (e.g. Picornaviruses, Togaviruses, Coronavirus including SARS-CoV-2). In some embodiments, the viral disease or viral infection is caused by viruses in the Baltimore classification Group V of viruses of single-stranded (antisense) RNA viruses (e.g. Orthomyxoviruses, Rhabdoviruses). In some embodiments, the viral disease or viral infection is caused by viruses in the Baltimore classification Group VI group of viruses of single-stranded (sense) RNA viruses with DNA intermediate in life-cycle (e.g. Retroviruses). In some embodiments, the viral disease or viral infection is caused by viruses in the Baltimore classification Group VII group of viruses of double-stranded DNA viruses with RNA intermediate in life-cycle (e.g. Hepadnaviruses).

In some embodiments, any of the vaccines disclosed herein in detail are used in the methods disclosed herein. In some embodiments, any of the APCs disclosed herein in detail are used in the methods disclosed herein. In some embodiments, the present disclosure provides a method of preventing or treating COVID-2019, or other viral diseases, in a subject, comprising any of the compositions disclosed herein. In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

In some embodiments, “treating” may encompass the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

In some embodiments, the subject's systemic immune response is increased or enhanced compared to the immune response of a subject not administered the present vaccine. The systemic response is sufficient for the subject to mount an immune response against the virus or virus antigen.

In some embodiments, a “prophylactic effect” is an inhibition of one or more symptoms associated with the viral infection for which the vaccine(s) are being administered.

In some embodiments, the terms “prevent,” “preventing,” “prevention,” “prophylactic treatment” and the like refer to reducing the probability of developing a disorder or condition (e.g., afflicted by viral infection) in a subject, who does not have, but is at risk of or susceptible to developing a disorder or condition (e.g., afflicted by viral infection).

In some embodiments, the terms “susceptible to” or “prone to” or “predisposed to” a specific disease or condition (e.g., viral infection) and the like may encompass a subject who based on genetic, environmental, health, and/or other risk factors is more likely to be infected by the virus than the general population. An increase in likelihood of being infected by the virus may be an increase of about 10%, 20%, 50%, 100%, 150%, 200%, or more.

As used herein, a “subject” refers in one embodiment, to a human or any other animal. In some embodiments, a subject is a healthy subject. In some embodiments, a subject refers to a human diagnosed with or displaying symptoms of a SARS-CoV-2 infection or COVID-19. A human includes pre- and postnatal forms. In one embodiment, subjects are humans being treated for symptoms associated with COVID-2019. In some embodiments, the subject is a human.

The immunogenic compositions and vaccines of the present disclosure are also advantageous to use to inoculate health care workers. In some embodiments, the subject is a health care worker.

In some embodiments, the vaccine is administered in the presence of adjuvants or carriers or other viral antigens. Furthermore, in some examples, treatment comprises administration of other agents commonly used against viral infection. Additionally, multiple, independently generated cells can be administered to a subject.

The term “therapeutically effective amount” or “effective amount” refers, in some embodiments, to a number of APCs sufficient to elicit a protective immune response in the subject to which it is administered. The immune response may comprise, without limitation, induction of cellular and/or humoral immunity.

The dosage regimen for treating a condition with the compositions or vaccines of present disclosure is selected in one embodiment, in accordance with a variety of factors, such as the type, age, weight, ethnicity, sex and medical condition of the subject, the infection or disease severity, and the particular formulation employed, and thus may vary widely while still be in the scope of the present disclosure.

Dosages may be titrated to optimize safety and efficacy. Typically, dosage-effect relationships from in vitro studies can provide useful guidance on the proper doses for patient administration. Studies in animal models can also be used for guidance regarding effective dosages for treatment of Coronavirus infection in accordance with the present disclosure.

In some embodiments, more than one administration of APCs can be delivered to the subject in a course of treatment. Dependent upon the particular course of treatment, multiple administrations may be given to a subject, with the administration repeated at various time intervals. For example, an initial or priming (or prime) administration may be followed by one or more booster (or boost) administrations. In some embodiments, the priming (or prime) and booster (or boost) administrations are delivered by the same route of administration and/or at about the same site. When multiple doses are administered, the first immunization dose may be higher than subsequent immunization doses.

In some embodiments, the APCs or vaccine is administered twice or more, 3 times, 4 times, 5 times, 6 times, 7 times, 8 times, 9 times, 10 times, 15 times, 20 times, 25 times, 30 times, 35 times, 40 times, 50 times, 60 times, 70 times, 80 times, 90 times or more. In some embodiments, the vaccine is administered at least once per week, at least twice per week, at least three times per week, at least four times per week, at least five times per week, at least six times per week, at least seven times per week. In some embodiments, the vaccine is administered at least once per day, at least twice per day, at least every eight hours, at least every four hours, at least every two hours, or at least every hour. In some embodiments, the present vaccine is administered for a duration of 1 day, 2 days, 3 days, 4 days, 5 days, 6 days, 7 days, 2 weeks, 3 weeks, 4 weeks, five weeks, six weeks, 2 months, 3 months, 4 months, 5 months, 6 months, 7 months, 8 months, 9 months, 10 months, 11 months, 12 months, 2 years, 3 years, 4 years, 5 years or more, or until the viral infection is treated.

In some embodiments, the APCs or vaccine may be administered once, twice, three times, four times, five times, six times, seven times, eight times, nine times, ten times, eleven times, twelve times, thirteen times, fourteen times, fifteen times, or more, within a regime to a subject/patient. In some embodiments, the APCs or vaccine may be administered every 2 days, every 3 days, every 4 days, every 5 days, every 6 days, every 7 days, every 8 days, every 9 days, every 10 days, every 11 days, every 12 days, every 13 days, every 14 days, every 16 days, every 18 days, every 21 days, every 1 month, every 2 months, every 3 months, every 6 months, every 1 year, every 2 years or at different frequencies

In some embodiments, any dose of APCs can be administered to the subject. In some embodiments, a dose between about 0.1×10¹⁰ APCs and 10¹⁰×10¹⁰ APCs are administered. In some embodiments, a dose between about 0.5×10¹⁰ APCs and 50×10¹⁰ APCs are administered. In some embodiments, a dose between about 1×10¹⁰ APCs and 10×10¹⁰ APCs are administered. In some embodiments, a dose of about 0.5×10¹⁰ APCs are administered. In some embodiments, a dose of about 1×10¹⁰ APCs are administered. In some embodiments, a dose of about 2.5×10¹⁰ APCs are administered. In some embodiments, a dose of about 5×10¹⁰ APCs are administered. In some embodiments, a dose of about 10×10¹⁰ APCs are administered. In some embodiments, a dose of about 25×10¹⁰ APCs are administered. In some embodiments, a dose of about 50×10¹⁰ APCs are administered.

In some embodiments, a boost vaccination can be administered 5-10 days after the prime vaccination; 10-15 days after the prime vaccination; 15-20 days after the prime vaccination; 20-25 days after the prime vaccination; 25-30 days after the prime vaccination; 30-40 days after the prime vaccination; 40-50 days after the prime vaccination; 50-60 days after the prime vaccination; 60-70 days after the prime vaccination; or at least 70 days after the prime vaccination.

In some embodiments, T cells can be removed from a subject and treated in vitro with the APCs, wherein the resulting CTLs are reinfused autologously or allogeneically to the subject. In some embodiments, the APCs of the present disclosure and the antigen-specific T lymphocytes generated with these APCs can be used as immunomodulating compositions for prophylactic or therapeutic applications for SARS-CoV-2 infection. In some embodiments, the APCs of the present disclosure can be used for generating CD8+ CTL, CD4+ CTL, and/or B lymphocytes for adoptive transfer to the subject. In some embodiments, antigen-specific CTLs can be adoptively transferred for therapeutic purposes in subjects afflicted with a viral infection.

It will be appreciated that APCs can be provided to the subject with additional active agents to achieve an improved therapeutic effect as compared to treatment with APCs alone. In some embodiments, additional active agents include anti-viral drugs.

In some embodiments, disclosed herein is a method for preventing or treating a SARS-CoV-2 infection in a subject comprising:
a) obtaining a population of peripheral blood mononuclear cells (PBMCs) from a subject;
b) isolating and culturing the APCs from the population of PBMCs;
c) contacting the APCs with a preparation of SARS-CoV-2 particles for a time period sufficient to generate APCs displaying at least two different SARS-CoV-2 antigens;
d) optionally isolating and expanding the APCs from step (c); and
e) administering the APCs to the subject.

In some embodiments, disclosed herein is a method for preventing or treating a SARS-CoV-2 infection in a subject comprising:
a) obtaining a population of peripheral blood mononuclear cells (PBMCs) from a subject;
b) isolating and culturing the APCs from the population of PBMCs;
c) contacting the APCs with mRNAs encoding SARS-CoV-2 antigens for a time period sufficient to generate APCs displaying at least two different SARS-CoV-2 antigens;
d) optionally isolating and expanding the APCs from step (c); and
e) administering the APCs to the subject.

In some embodiments, the present disclosure provides a method of preventing and/or treating SARS-CoV-2 infection in a subject, the method comprising the step of administering to the subject the vaccine described herein in detail.

In some embodiments, the present disclosure provides a method of eliciting an immune response to SARS-CoV-2 in a subject, the method comprising the step of administering to the subject the vaccine described herein in detail.

### COVID-19

In some embodiments, a viral disease or viral infection comprises COVID-19. In some embodiments, a viral disease is caused by SARS-CoV-2. A skilled artisan will recognize that COVID-19, also termed “novel coronavirus pneumonia”, “NCP”, “SARS-CoV-2 acute respiratory disease”, and “COVID-19” comprises an infectious respiratory disease caused by the 2019 novel coronavirus (SARS-CoV-2), which was first detected during the 2019-20 Wuhan coronavirus outbreak. In some embodiments, SARS-CoV-2 is transmitted through human-to-human transmission, generally via respiratory droplets as sneeze, cough or exhalation. In some embodiments, COVID-19 symptoms appear after an incubation period of between 2 to 14 days. In some embodiments, coronavirus primarily affects the lower respiratory tract. In some embodiments, coronavirus primarily affects the upper respiratory tract. In some embodiments, COVID-19 symptoms comprise fever, coughing, shortness of breath, pain in the muscles, tiredness, pneumonia, acute respiratory distress syndrome, sepsis, septic shock, death, or any combination thereof.

A skilled artisan will recognize that SARS-CoV-2 belongs to the broad family of viruses known as coronaviruses. SARS-CoV-2 is a positive-sense single-stranded RNA (+ssRNA) virus. SARS-CoV-2 is a member of the subgenus Sarbecovirus (Beta-CoV lineage B), having an RNA sequence of approximately 30,000 bases in length.

Eighty-one genomes of SARS-CoV-2 had been isolated and reported. The present disclosure comprises compositions and methods for treating these SARS-CoV-2 variants, or any further one.

A skilled artisan will recognize that seven coronavirus types are known to affect humans. The compositions and methods disclosed herein are useful for treating any of them. In some embodiments, coronavirus comprises Human coronavirus 229E (HCoV-229E). In some embodiments, coronavirus comprises Human coronavirus OC43 (HCoV-OC43). In some embodiments, coronavirus comprises Severe acute respiratory syndrome-related coronavirus (SARS-CoV). In some embodiments, coronavirus comprises Human coronavirus NL63 (HCoV-NL63, New Haven coronavirus). In some embodiments, coronavirus comprises Human coronavirus HKU1. In some embodiments, coronavirus comprises Middle East respiratory syndrome-related coronavirus (MERS-CoV), previously known as novel coronavirus 2012 and HCoV-EMC. In some embodiments, coronavirus comprises Novel coronavirus (SARS-CoV-2), also known as Wuhan coronavirus.

In some embodiments, the APCs and compositions disclosed herein are used for treating a virus other than SARS-CoV-2.

The following examples are presented in order to more fully illustrate the preferred embodiments of the present disclosure. They should in no way be construed, however, as limiting the broad scope of the disclosure.

### EXAMPLES

### Statistical Analysis

In the following examples, a one-way ANOVA statistical analysis, followed by Bonferroni's correction, was performed on pooled replicates of 3-7 independent experiments (donors). For unpaired Student's t-test analyses, P values smaller than 0.05 were considered significant, with all tests being two-tailed. Outlier values were excluded using the Graphpad outlier calculator.

### Example 1—Dendritic Cell Infection with Chemically-Inactivated SARS-CoV-2

### Preparations of Dendritic Cells (DCs)

DCs were prepared from PBMCs, first isolated from a fresh apheresis sample, followed by purification of CD14+ monocytes by positive selection beads. Isolated monocytes were seeded in T75 flasks in the presence of differentiation cytokines for 7 days (GM-CSF and IL-4). During the differentiation period, the medium was refreshed twice-on days 3 and 6 with maturation factors added on day 7. In addition, control group cells were harvested, and the expression of typical DC cluster of differentiation (CD) markers were analyzed by flow cytometry. On day 8, matured cells were harvested, and CD marker expression was analyzed again and compared to expression pattern on day 7.

Transduction with Chemically-Inactivated SARS-CoV-2

Chemically-inactivated (inactivated by chemical treatment) SARS-CoV-2 was used (Cat. No. 9101SD Inactivated SARS-CoV-2 Isolate USA-WA1/2020 109 genome copies/ml, 500 μL tube, by Microbiologics). For the RT-qPCR reaction, the negative control was ultra-pure RNase-free water, whereas the positive control contained synthetic target RNA molecules with sequences of the viral N and E gene targets, as well as the RNase P gene target.

Human DCs were transduced with the chemically-inactivated SARS-CoV-2 for 48 hours either with or without the transduction reagent polybrene (PB), at a multiplicity of infection (MOI) of 1 or 2, in differentiation media or maturation media.

Differentiation media includes:
cAIM media+50 ng/mL human recombinant GM-CSF+40 ng/mL human recombinant IL-4.

Maturation media includes:
cAIM media+10 ng/mL human recombinant IL-1beta+100 ng/mL human recombinant IL-6+25 ng/ml human recombinant TNF-α+1 μg/mL CpG+10 μg/mL PGE2+50 ng/ml LPS.

Viral envelope and nucleocapsid gene expression analyses served as measured readouts. After a 48-hour incubation of DCs with the chemically-inactivated SARS-CoV-2, RNA was extracted from the cells using Quick-RNA™ Viral Kit (Zymo Research). This was followed by a quantitative real-time PCR (qRT-PCR), employing the Tamix-Go CVL SARS-CoV-2 (SC2) Multiplex One-Step RT-qPCR detection kit. The Tamix-Go SARS-CoV-2 kit is designed for in vitro diagnostic testing, based on RT-qPCR technology for qualitative detection of the SARS-CoV-2 coronavirus ribonucleic acid (RNA). The primers and probes mix of this kit target specific conserved regions within the viral Nucleocapsid (N) and Envelope (E) genes, using the HEX and FAM channels, respectively. The reaction mix also detects the RNase P gene, which serves as an internal control, to confirm the integrity of the sampling and the RNA extraction process, using the Cy5 channel. The relative gene expression of the viral envelope and nucleocapsid was measured.

### Results

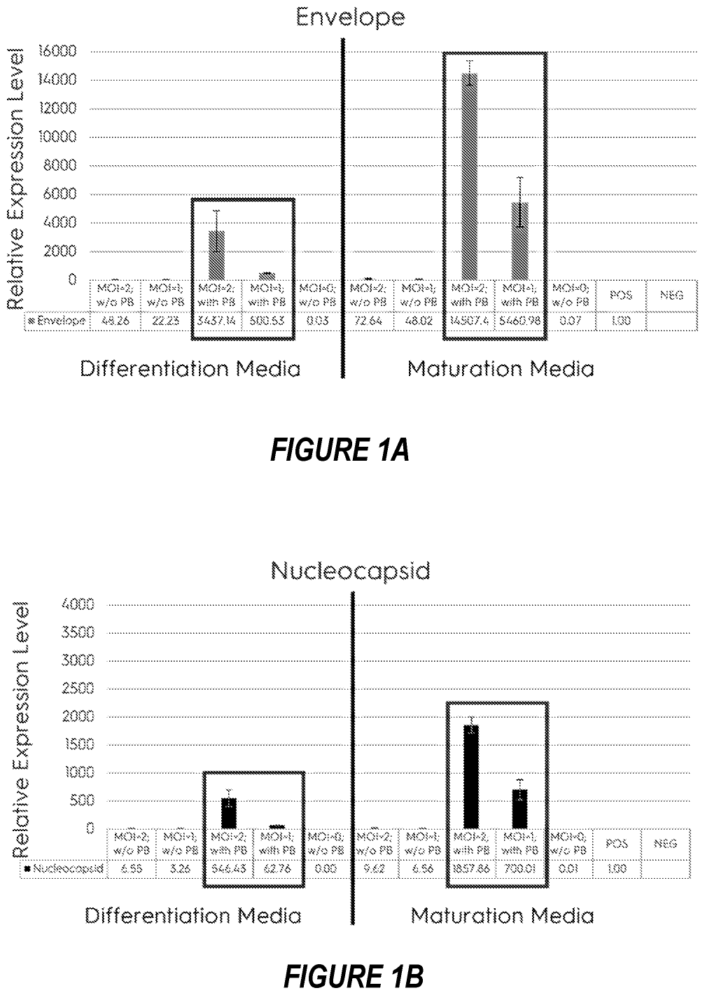

A substantial expression of the viral envelope and nucleocapsid genes was observed in SARS-CoV-2-transduced DCs, indicating a successful production of multi-antigenic-expressing cells (FIG. 1A, B).

The expression of both viral envelope and nucleocapsid genes was optimal when using an infection enhancer, such as polybrene, and maturation media conditions (maturated DCs), and an MOI of 2 (FIG. 1A, B, frames).

### Example 2—T Cell Activation by Dendritic Cells Transduced with Chemically-Inactivated SARS-CoV-2

To further evaluate the efficacy of SARS-CoV-2 multi-antigenic-displaying DCs, cells were exposed to donor-matched PBMCs, to measure immune activation by flow cytometry, looking at specific known T cell activation/phenotypic markers (e.g. CD4, CD8, CD25, CD69, CD137).

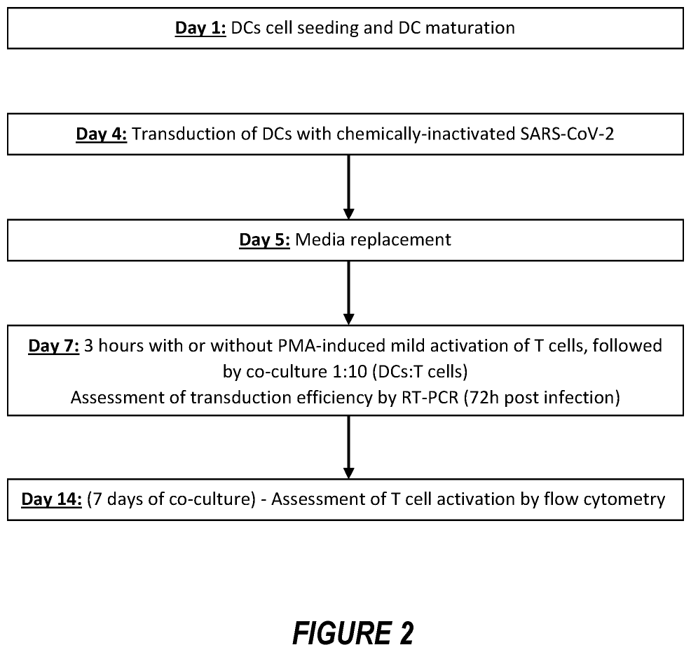

The study included DCs infected with the chemically-inactivated SARS-CoV-2 at an MOI of 2.5 for 72 hours. After washing the chemically-inactivated SARS-CoV-2 from culture, T cells were incubated with DCs for additional 7 days, as shown in the study flow in FIG. 2.

On Day 1, DCs were seeded and maturated. On Day 4, DCs were transduced/infected with the chemically-inactivated SARS-CoV-2, followed by media replacement on Day 5. On Day 7, T cells were activated for 3 hours (non-activated T cells serve as a negative control) and were then exposed to DCs. Transduction efficiency was assessed by qRT-PCR, 72 hours post infection. After 7 days of co-culture, T cell activation was assessed by flow cytometry (FACS). Briefly, for FACS analysis, cell media was discarded, and cells were washed twice with PBS. Then, cells were collected in cAIM media and counted. This was followed by cell staining using different antibodies in FACS buffer containing BSA, and corresponding isotype controls for each, including FITC anti-CD4, APC/Fire™ 750 anti-CD8a, APC anti-CD25, PE anti-CD137 (4-1BB), and a brilliant violet 421™ anti-CD69. Subsequently, samples were analyzed by the Fortessa FACS analyzer. Different treatment groups are shown in Table 1.

TABLE 1

| | | | Chemically- | |
|---|---|---|---|---|
| | | | inactivated | |
| Group | DC Cell | | SARS-CoV-2 | |
| No. | Number | Polybrene | (MOI = 2.5) | T Cells |
| 1 | — | − | — | Naïve |
| 2 | — | − | — | Mildly-activated - |
| | | | | 3 h- PMA activation |
| | | | | cocktail mix |
| 3 | 100,000 | + | Non-infected | Naïve |
| 4 | 100,000 | + | Non-infected | Mildly-activated - |
| | | | | 3 h PMA- activation |
| | | | | cocktail mix |
| 5 | 100,000 | + | Infected | Naïve |
| | | | (MOI = 2.5) | |
| 6 | 100,000 | + | Infected | Mildly-activated - |
| | | | (MOI = 2.5) | 3 h PMA- activation |
| | | | | cocktail mix |

### Results

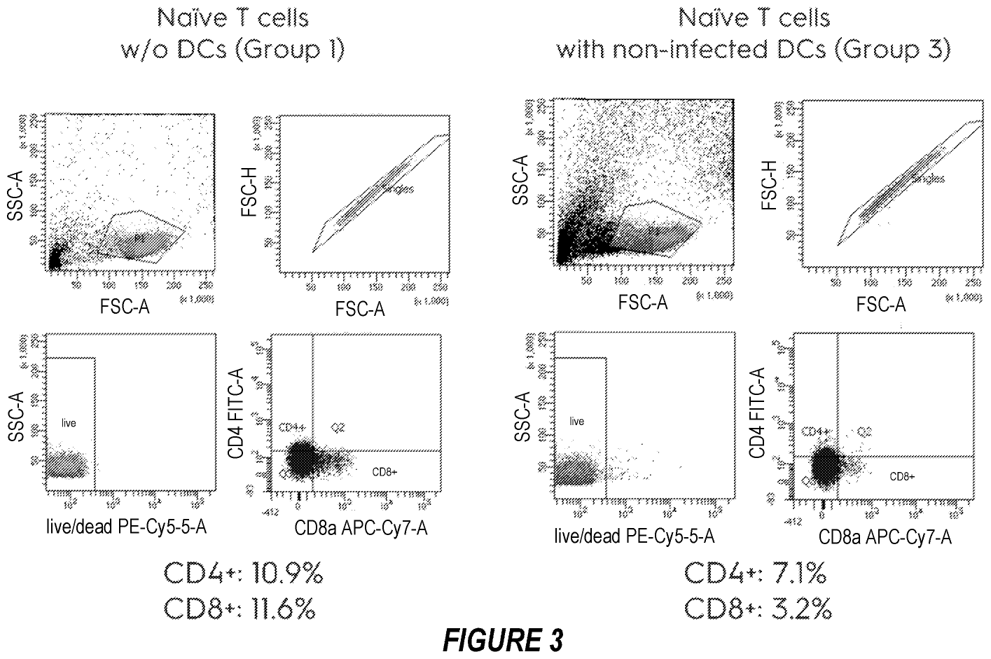

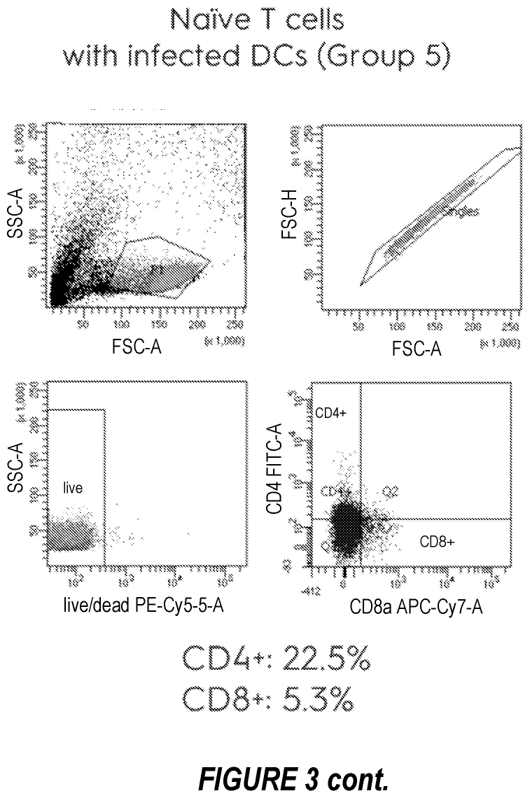

The percentage of naïve CD4+ T cells was increased when exposed to chemically-inactivated SARS-CoV-2-infected DCs. Naïve T cells exposed to infected DCs (group 5) displayed an increase in the CD4+ T cell population (22.5%), compared with naïve T cells incubated without DCs (10.9%, group 1), or naïve T cells incubated with non-infected DCs (7.1%, group 3). No substantial differences were observed in the CD8+ T cell population among the 3 groups (FIG. 3).

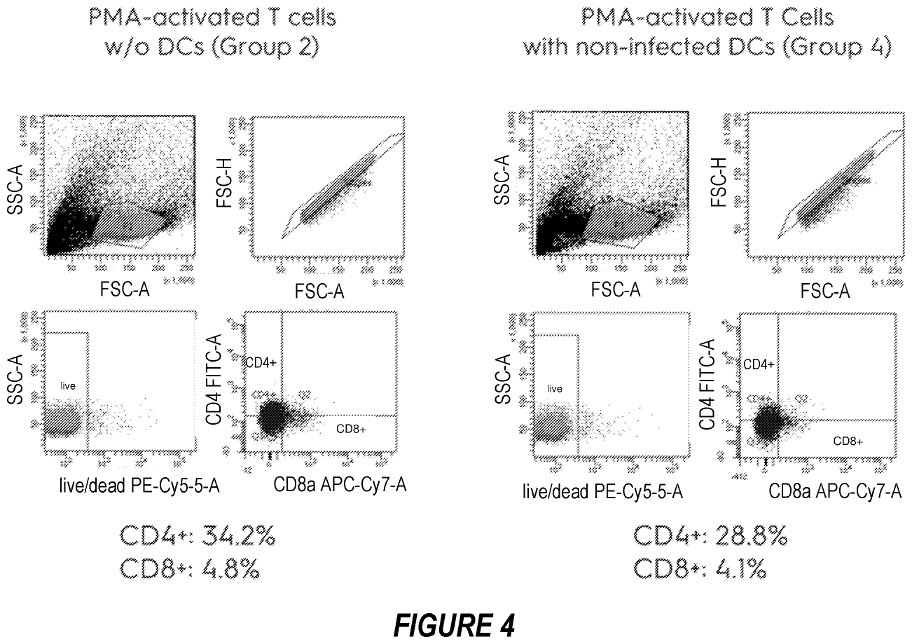

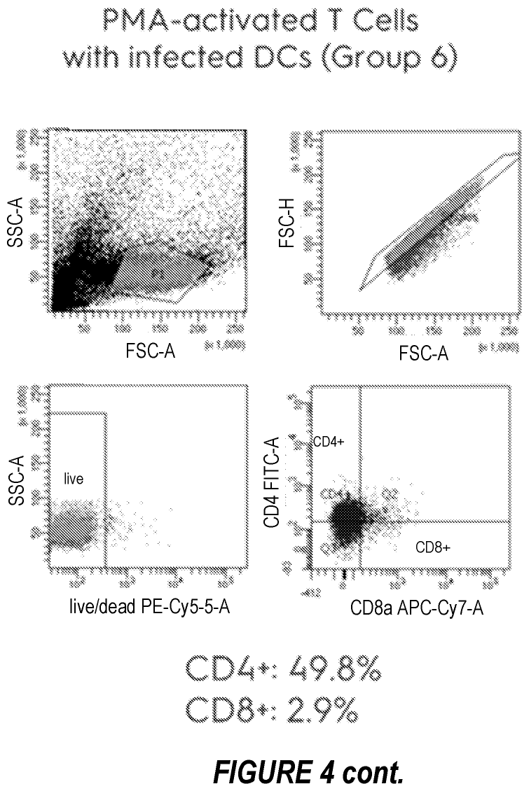

The percentage of Phorbol 12-myristate 13-acetate (PMA)-activated CD4+ T cells was increased when exposed to chemically-inactivated SARS-CoV-2-infected DCs. PMA-activated T cells exposed to infected DCs (group 6) displayed an increase in the CD4+ T cell population (49.8%), compared with PMA-activated T cells incubated without DCs (34.2%, group 2), or PMA-activated T cells incubated with non-infected DCs (28.8%, group 4). No substantial differences were observed in the CD8+ T cell population among the 3 groups (FIG. 4).

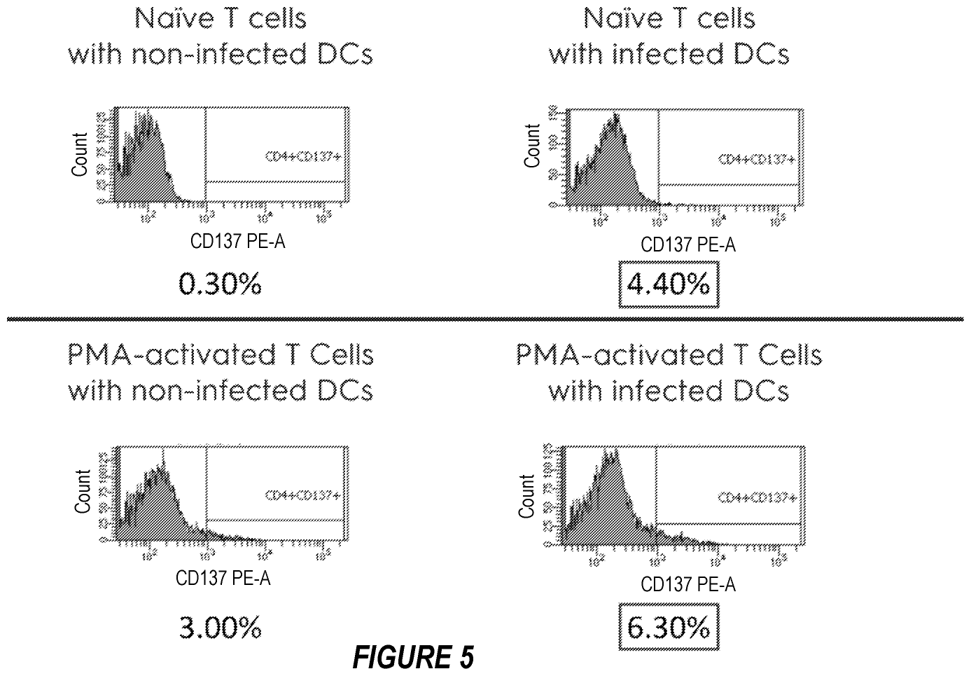

Activated T cell sub-populations were increased following exposure to chemically-inactivated SARS-CoV-2-infected DCs. An activated T cell sub-population was analyzed by CD4+/CD137+ marker expression using FlowJo program; the gating strategy included the selection of the live cell population, followed by selection of single cells. Out of the single cell population, the CD4+/CD8a− cells were identified. Then, the CD137+ cell percentile was calculated out of the CD4+/CD8a− cells. An increase in the CD4+/CD137+ cell population was displayed for either naïve (4.40%) or PMA-activated (6.30%) T cells, exposed to infected DCs. This is in comparison to naïve (0.30%) or PMA-activated (3.00%) T cells, exposed to non-infected DCs. In addition, lower T cell population percentages were also observed for Naïve (0.20%) or PMA-activated (4.70%) T cells incubated without DCs (FIG. 5, Table 2).

TABLE 2

| Group Description | CD4+ | CD4+/CD137+ |
|---|---|---|
| Naïve T cells w/o DCs | 10.90% | 0.20% |
| Naïve T cells with non-infected DCs | 7.10% | 0.30% |
| Naïve T cells with infected DCs | 22.5% | 4.40% |
| PMA-activated T cells w/o DCs | 34.20% | 4.70% |
| PMA-activated with non-infected DCs | 28.80% | 3.00% |
| PMA-activated T cells with infected DCs | 49.80% | 6.30% |

### CONCLUSIONS

CD4+ T cell percentage was increased when pre-exposed to chemically-inactivated SARS-CoV-2-infected DCs, indicating a proliferation of the CD4+ T cell subset population.

CD4+/CD137+ T cell percentage was increased when pre-exposed to chemically-inactivated SARS-CoV-2-infected DCs, indicating activation of the CD4+/CD137+ T cell subset. No substantial differences were observed in CD8 expression among groups.

The SARS-CoV-2 multi-antigenic-displaying DCs were able to efficiently activate T-cells.

### Example 3—SARS-CoV-2-Transduced DCs Significantly Express SARS-CoV-2 Target Genes, are Immunogenic, and Increase T Cell Functionality

### Experimental Outline:

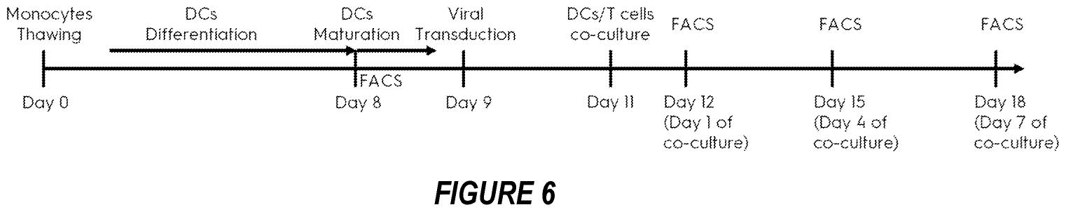

DC differentiation and maturation was performed between Day 0 and Day 8, with maturated DCs characterized on Day 8 by Fluorescence-activated cell sorting (FACS), examining specific DC cell markers. On Day 9, viral transduction/infection with the chemically-inactivated SARS-CoV-2 (ciSARS-CoV-2) virus was performed for 48 hours. On Day 11, SARS-CoV-2 transduced DCs were exposed to donor-matched enriched T cell population in a co-culture till Day 18. During the co-culture period, on Day 12 (Day 1 of co-culture), Day 15 (Day 4 of co-culture), and Day 18 (Day 7 of co-culture), FACS was performed for several T cell markers, to examine cell immunogenicity, and activation kinetics throughout the co-culture period. The experimental outline, including DC differentiation and maturation, viral transduction, co-culturing with T cells, and FACS analysis on Days 1, 4, and 7 of the co-culture is presented in FIG. 6. The experimental groups are presented in Table 3.

TABLE 3

| | | | ciSARS-CoV-2 | |
|---|---|---|---|---|
| Group No. | DC Cell No. | Polybrene | (MOI = 2.5) | T Cells |
| 1 | — | − | — | Naïve |
| 2 | 100,000 | + | Non-transduced | (1 × 10⁶) |
| 3 | 100,000 | + | Transduced | |
| | | | (MOI = 2.5) | |

### Results:

### Dendritic Cell (DC) Characterization on Day 7 of In Vitro Differentiation:

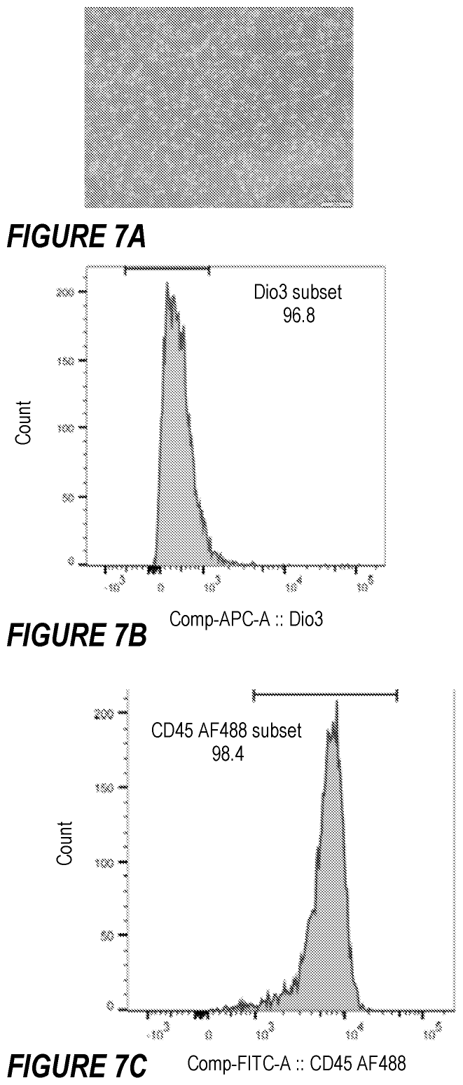

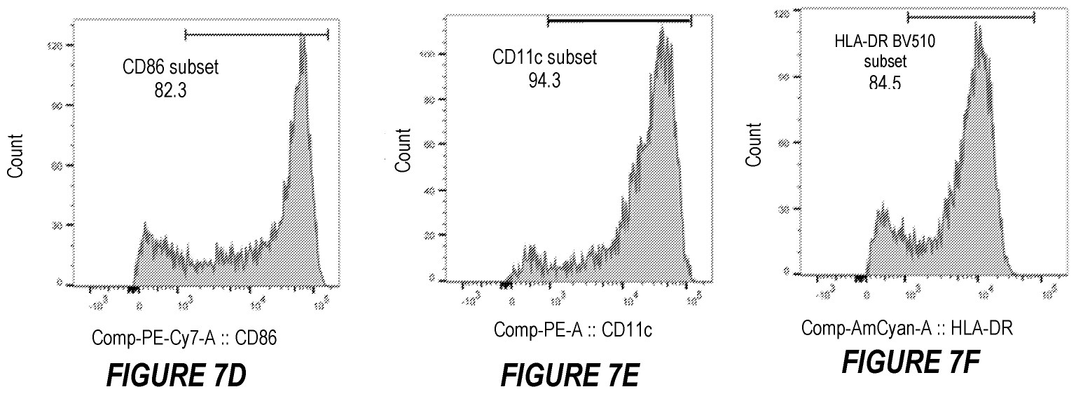

Dendritic cells (DCs) were differentiated in vitro from isolated CD14+ monocytes, using a 7-day differentiation protocol. As part of cell characterization, cell morphology and markers were analyzed by FACS. Upon monocyte thawing, an average of 95% cell viability was observed. As evident from the data presented in FIGS. 7A-7F, after 7 days of differentiation, the morphology of the cells was consistent with DC morphology (FIG. 7A), and the cells expressed DC specific cell markers (Table 4), as well as a cell viability of >80%. On Day 8, following maturation, DCs were collected, counted, and characterized by FACS analysis, and similarly to Day 7, also exhibited an average of >80% cell viability (FIGS. 7B-7F).

Table 4 shows the average percentage of specific cell markers of three DC cultures isolated from different donors. The vast majority of viable DCs (Dio3 negative cells) were positive to CD45+ marker (>99%).

TABLE 4

| | Positive cells |
|---|---|
| Marker | (% of parental) |
| Life | 90.54 ± 7.2% |
| CD45+ | 99.87 ± 0.1% |
| CD45+/ CD11+ | 95.58 ± 7.2% |
| CD45+/ CD86+ | 76.97 ± 8.9% |
| CD45+/HLA-DR+ | 80.50 ± 13.2% |

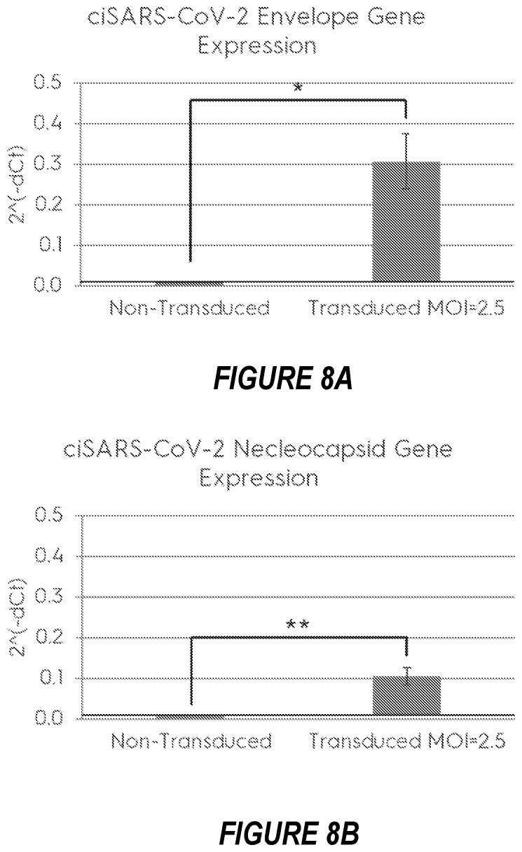

DCs transduced with the ciSARS-CoV-2 exhibit expression of viral genes, such as the envelope and nucleocapsid. qRT-PCR, comparing the experimental groups of non-transduced DCs vs. transduced DCs at an MOI of 2.5, showed a significant viral gene expression in transduced cells, compared with non-transduced cells (FIGS. 8A-8B). Importantly, following cell transduction with the ciSARS-CoV-2, cell morphology and viability was preserved with >80% cell viability.

### Implemented FACS Gating Strategy

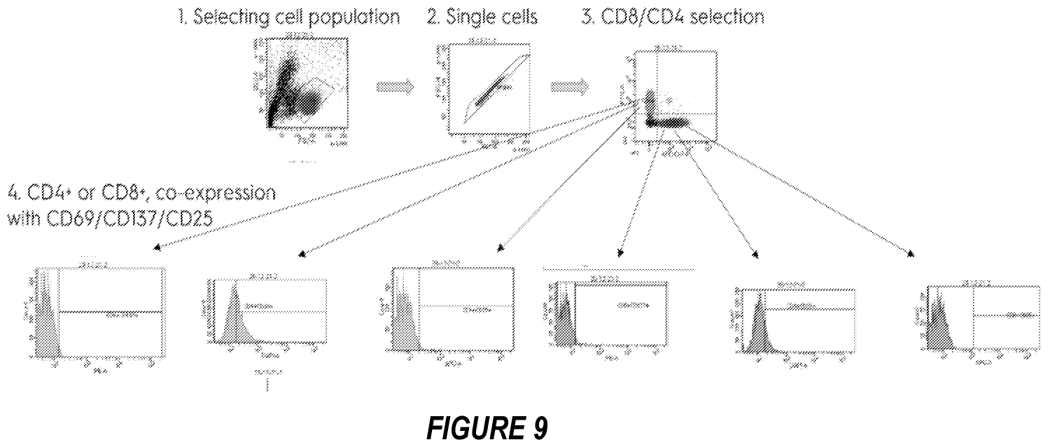

The implemented FACS gating strategy was comprised of several stages, as shown in FIG. 9. First, the cell population was selected, followed by single cell selection, and CD4+/CD8+ cell population selection. Then, for either CD4+ or CD8+ cell populations, cells co-expressing CD69/CD137/CD25 were further selected and analyzed.

### Characterization of Enriched T Cell Population:

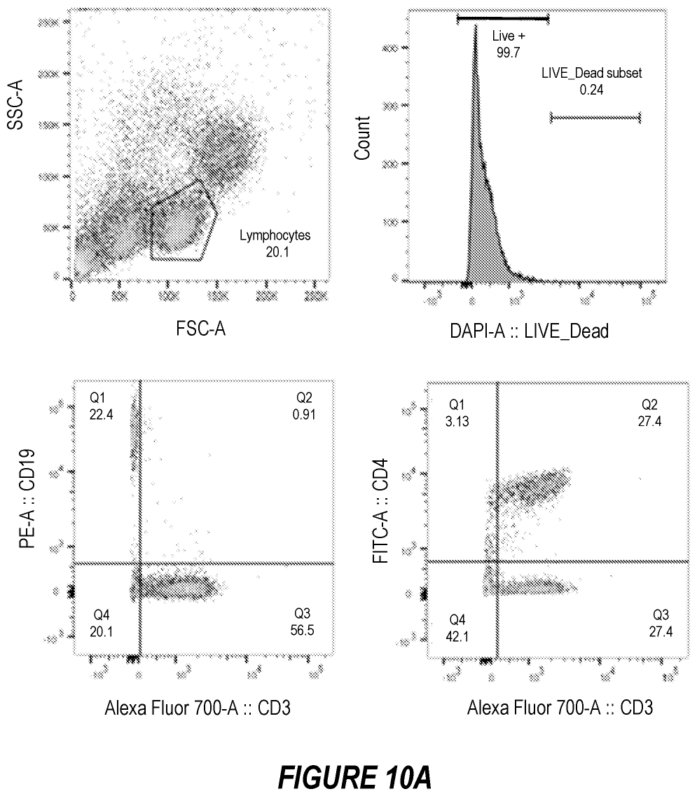

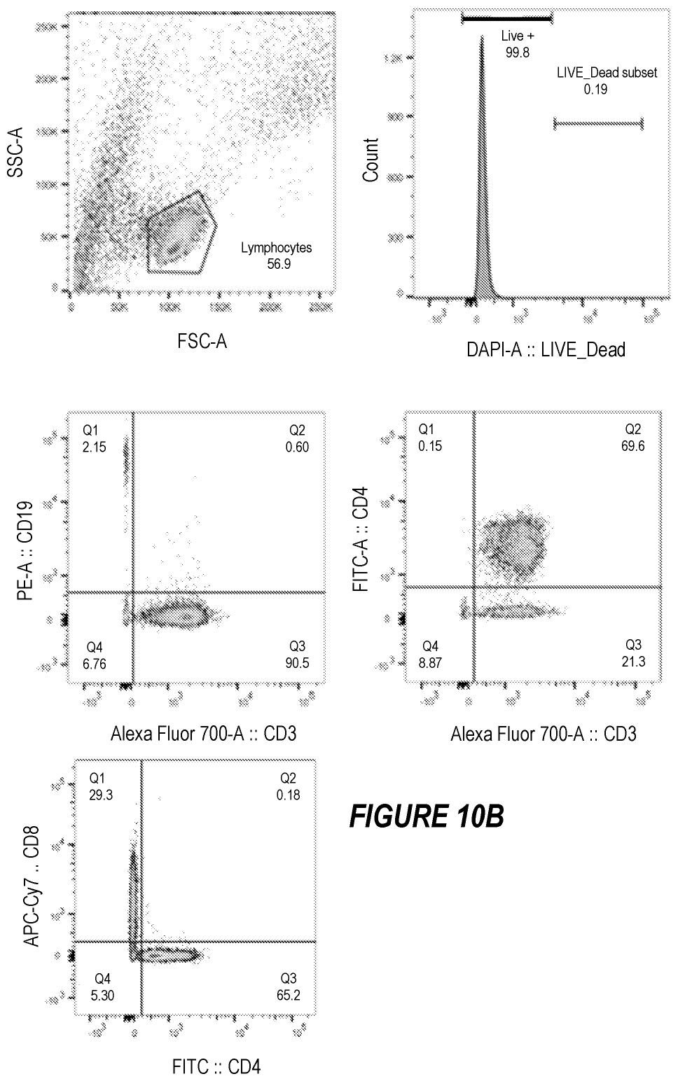

An enriched T cell population was employed as a tool for analyzing the ability of infected DCs to activate the immune response. The expression of specific T cell markers in total PBMCs (FIG. 10A) was analyzed and compared with the results of an enriched T cell population from the same donor (FIG. 10B). First, lymphocyte viability was analyzed by FACS, and within the viable lymphocyte population, the percentage of T cells was analyzed using the CD3 marker, and the percentage of B cells was analyzed using the CD19 marker. Within the CD3+/T cell population, the percentages of the CD4+ and CD8+ cell populations were determined.

As shown in FIGS. 10A-10B, 90% of all analyzed cells in the enriched T cell population were CD3+ T cells, compared with only 56% in the PBMCs (CD3+). The percentage of B cells in the enriched T cell population was low, with only 10% of the population in total PBMCs (2.15 vs. 22% in PBMCs, CD19+). Among the CD3+ T cells, approximately ⅔ were CD4+, and ⅓ were CD8+. No CD4+ cells were CD3− T cells.

### CD25+ T Cell Population on Days 1, 4, 7 of Co-Culturing

CD25 is expressed constitutively on the surface of several subsets of peripheral blood lymphocytes, such as regulatory and resting memory T cells, and is considered to be the most prominent cellular activation marker. CD25 is upregulated within 24 hours of stimulation of the T Cell Receptor (TCR)/CD3 complex and remains elevated for a few days.

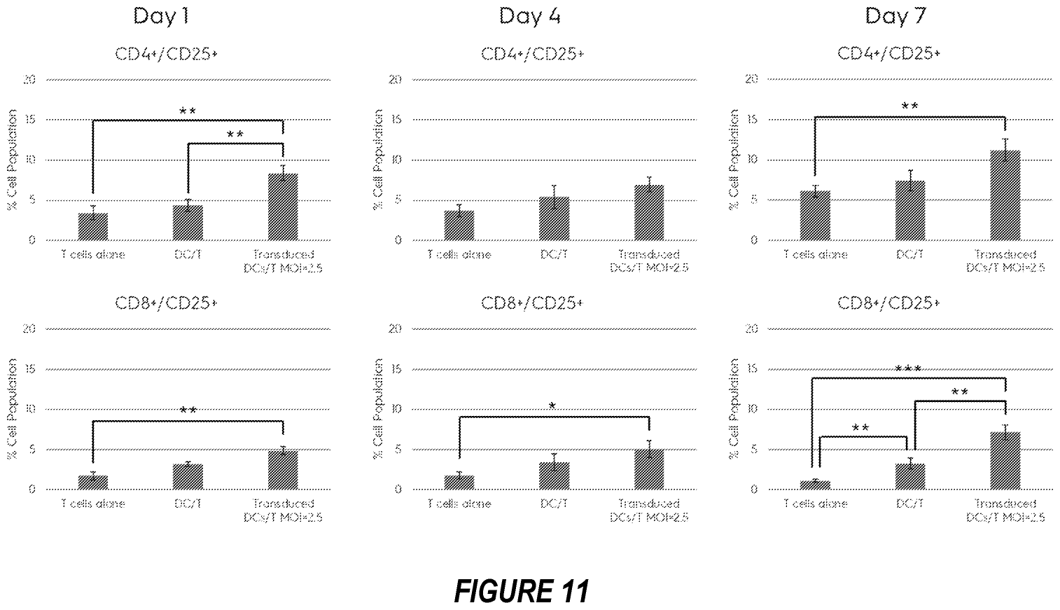

As shown in FIG. 11, when exposed to SARS-CoV-2-transduced DCs, CD25 expression was significantly increased on Day 1 for CD4+ and CD8+ cells, compared with control T cells incubated alone (**p<0.01), and T cells incubated with non-transduced DCs for CD4+ cells only (**p<0.01). On Day 4, for CD4+ cells, an elevated CD25 expression trend was observed, whereas for CD8+ cells, incubated with transduced DCs, CD25 expression was significantly increased, compared with control T cells incubated alone (*p<0.05). On Day 7, a significant increased CD25 expression was displayed for CD4+ and CD8+ cells, incubated with transduced DCs vs. T cells alone (**p<0.01, ***p<0.001), and T cells incubated with non-transduced DCs in the case of CD8+ cells only (**p<0.01). Additionally, for CD8+ cells, CD25 expression was significantly increased for T cells incubated with non-transduced DCs, compared with T cells incubated alone (**p<0.01).

### CD69+ T Cell Population on Days 1, 4, 7 of Co-Culturing

CD69 is a membrane-bound, type II C-lectin receptor, serving as a classical early marker of lymphocyte activation due to its rapid appearance on the surface of the plasma membrane after stimulation. Importantly, depending on stimulation, high expression levels of CD69 may also be found between 96-120 hours.

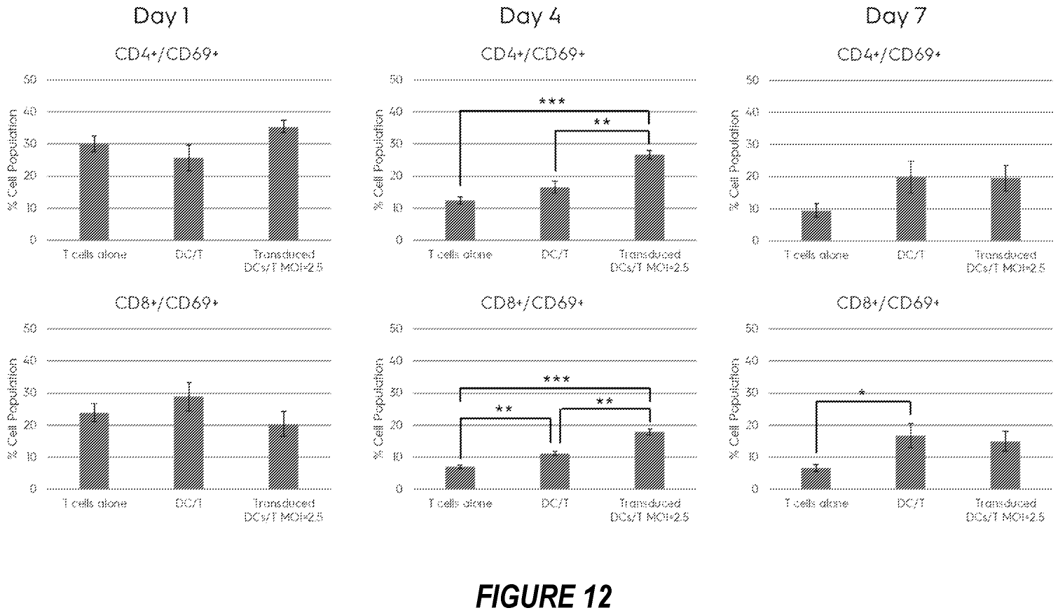

As shown in FIG. 12, when exposed to SARS-CoV-2-transduced DCs, CD69 expression was significantly increased on Day 4 (96 hours post exposure to stimulation) for CD4+ and CD8+ cells, compared with control T cells incubated alone (***p<0.001), and T cells incubated with non-transduced DCs (**p<0.01). In addition, CD8+ T cells exposed to non-transduced DCs also exhibited significant increase in CD69 expression, compared with T cells alone (**p<0.01). On Day 7, although not significant, higher CD69 expression trends were observed for CD4+ cells, exposed to DCs, compared with T cells alone. For CD8+ cells, CD69 expression was significantly increased in cells exposed to non-transduced DCs, compared with T cells alone (*p<0.05).

### CD137+ T Cell Population on Days 1, 4, 7 of Co-Culturing

CD137 (4-1BB) was originally identified as a molecule expressed on activated mouse and human CD8+ and CD4+ T cells. It is a member of the TNFR family and mediates costimulatory and antiapoptotic functions, promoting T-cell proliferation and T-cell survival. CD137 has been reported to be up-regulated—depending on the T-cell stimulus—from 12 hours to up to several days after stimulation.

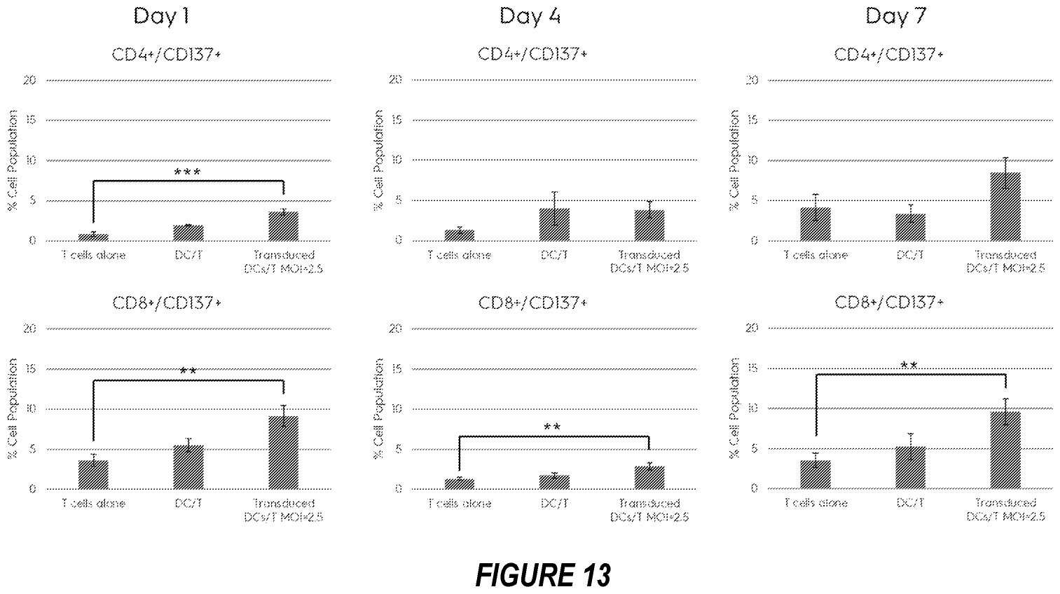

As shown in FIG. 13, upon exposure to SARS-CoV-2-transduced DCs, CD137 expression was significantly increased on Day 1 for both CD4+ and CD8+ cells (**p<0.01, ***p<0.001). On Days 4 and 7, CD137 expression was significantly increased in CD8+ T cells only, exposed to transduced DCs, compared with T cells alone (**p<0.01).

### SARS-CoV-2-Transduced DCs Activate T Cells

The increased CD25/CD69/CD137 cell markers (FIGS. 11-13) demonstrate that SARS-CoV-2-transduced DCs activate T cells.

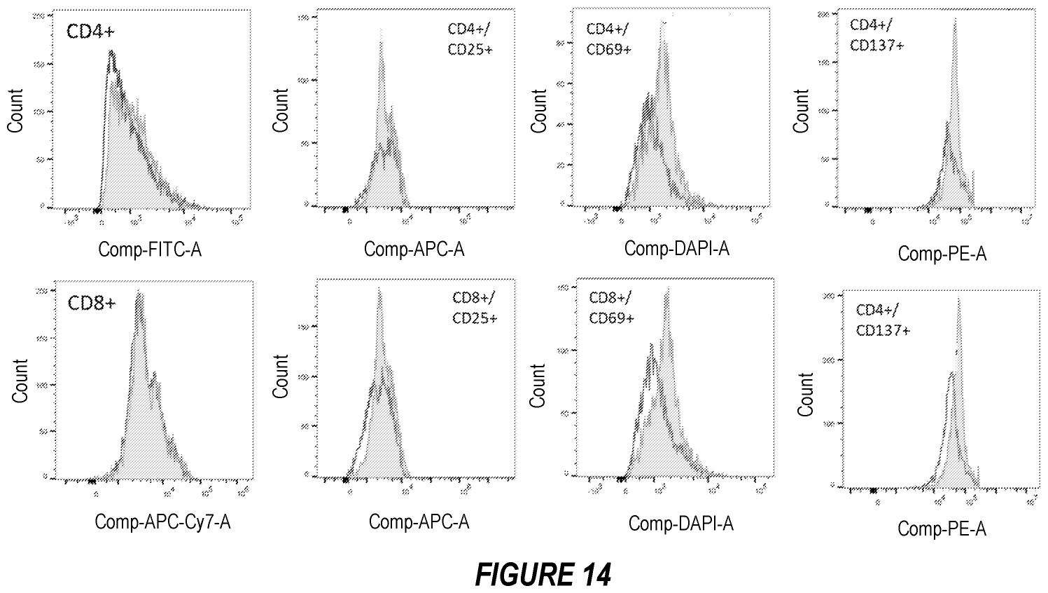

SARS-CoV-2-transduced DCs were co-cultured with enriched T cell population for 7 days. As shown in FIG. 14, T cells were activated in the co-culture, as indicated by the activation of the specific T cell markers, including CD25, CD69, and CD137.

### Total CD4+ and CD8+ T Cell Populations on Days 1, 4, 7 of Co-Culturing

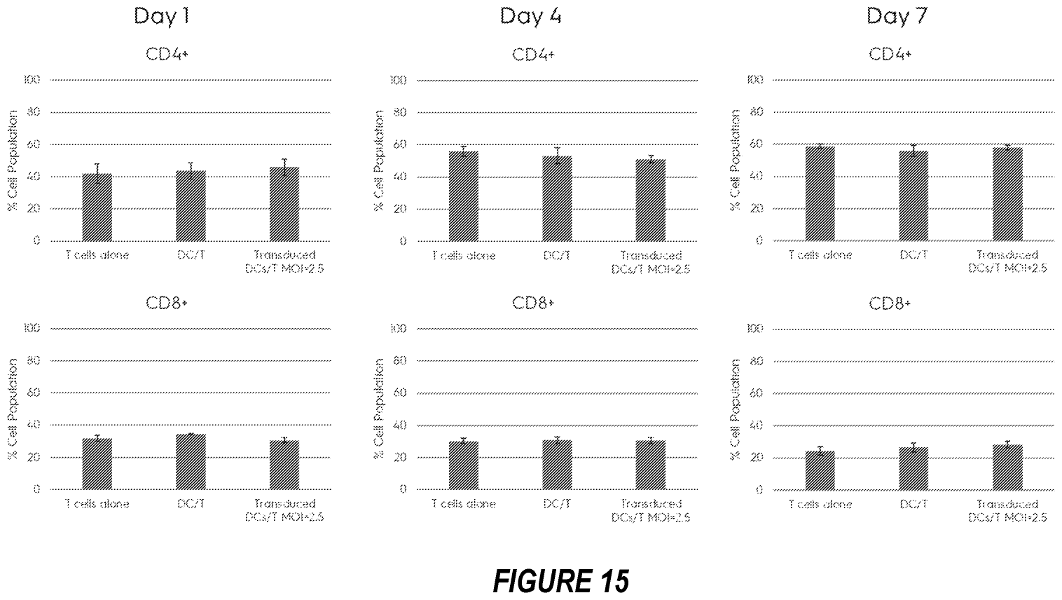

No alterations were found for the total CD4+ and CD8+ cell populations among the experimental groups of T cells alone, non-transduced DCs co-cultured with T cells, and transduced DCs co-cultured with T cells, on days 1, 4, and 7 (FIG. 15). These findings suggest that under the conditions described above, T cells are activated (as also demonstrated using the activation markers CD25/CD69/CD137 above).

### TNFα Secretion on Days 1, 4, 7 of Co-Culturing

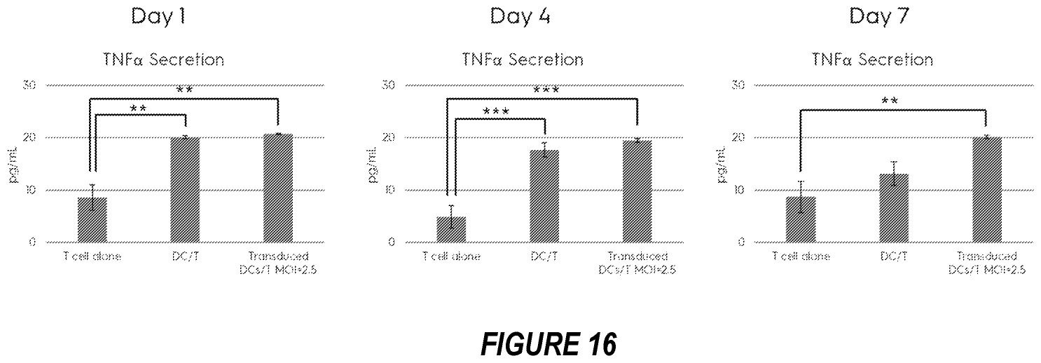

Tumor Necrosis Factor (TNF) a is a pleiotropic cytokine involved in the pathogenesis of a range of physiological processes that control inflammation, anti-tumor responses, and immune system homeostasis. TNFα is best known for its protective activity against pathogens, being a product of effector CD4 and CD8 T cells or innate cells, that can lead to killing of infected cells. FIG. 16 presents the results of media TNFα secretion among the experimental groups of T cells alone, as well as non-transduced, and transduced DCs exposed to T cells.

Specifically, naïve T cells incubated alone, and not exposed to DCs, exhibited a basal TNFα secretion level. T cells exposed to SARS-CoV-2-transduced DCs, exhibited a significant increase in media TNFα secretion levels from Day 1, throughout Day 4, and till Day 7, and remained high during this time period (˜20 μg/mL). Interestingly, T cells exposed to non-transduced DCs displayed a significant increase in TNFα secretion levels on Day 1, which decreased on Day 4, yet was still significant, and further decreased on Day 7, to non-significant levels, compared with basal TNFα secretion level of T cells alone.

These results indicate that T cells are substantially activated in the presence of DCs, an effect which is preserved especially when co-cultured with SARS-CoV-2-transduced DCs, compared with non-transduced DCs. Interestingly, CD137 upregulation serves as a surrogate marker for specifically activated CD8+ T cells in vitro and ex vivo, with excellent correlations of CD137 expression and TNFα cytokine production. Accordingly, the finding of increased TNFα secretion levels is in line with the CD137 expression results shown in FIG. 13.

### Conclusions:

DCs transduced with a ciSARS-CoV-2 virus exhibited immunogenicity, as shown by the significant increase of the T cell activation markers CD25, CD69, and CD137, without altering total CD4+ and CD8+ cell populations. These findings were further complemented by similar secretion patterns of TNFα, suggestive of increased T cell functionality.

### Example 4—T Cell Activation In-Vivo Following Vaccination with Dendritic Cells Transduced with Chemically-Inactivated SARS-CoV-2

To assess in vivo immunogenic response of infected DCs, 2-month-old male B6.Cg-Tg (K18-hACE2)2Prlmn/J mice will be administered intradermally once with mouse infected DCs (mice will be grouped for various doses). The animals will be evaluated for morbidity and mortality on a daily basis, with clinical observation and body weight monitoring during acclimation and before dosing. On days 7 and 14 after administration (4 animals in each termination point)—blood and lymph structures will be harvested and evaluated for anti-SARS-CoV-2 antibodies (IgM and IgG) using ELISA assay and immune cell prevalence (mainly CD4 and CD8) by Flow cytometry.

To establish the in vivo mouse model, 2-month-old male B6.Cg-Tg (K18-hACE2)2Prlmn/J mice will be administered intranasally once, with several doses of SARS-CoV-2. The animals will be evaluated for morbidity and mortality on a daily basis, with clinical observation and body weight monitoring during acclimation and before dosing. On days 3, 7 and 14 after virus administration (3 animals in each termination point)—lungs and snout will be harvested, and bronchoalveolar lavage will be performed to examine viral load.

In in vivo feasibility and proof-of-concept studies, 2-month-old male B6.Cg-Tg (K18-hACE2)2Prlmn/J mice will be administered intradermally with several infected DC doses and a determined SARS-CoV-2 dose intranasally, based on the aforementioned immunogenicity and model establishment studies. The animals will be evaluated for morbidity and mortality on a daily basis, with clinical observation and body weight monitoring during acclimation and before dosing. On days 7, 14 and 21 after virus administration (4 animals in each termination point)—blood and lymph structures will be harvested and evaluated for antibodies (IgM and IgG) using ELISA assay and immune cell prevalence (mainly CD4 and CD8) by Flow cytometry, snout and lungs will be evaluated for viral load in addition to bronchoalveolar lavage.

1. A multi-antigenic autologous, cell-based SARS-CoV-2 vaccine, said vaccine comprising autologous antigen presenting cells (APCs) displaying at least two different SARS-CoV-2 antigens.

2. The vaccine of claim 1, wherein said APCs are activated, and wherein said APCs comprise dendritic cells, macrophages, B cells, monocytes or a combination thereof.

3. The vaccine of claim 1, wherein said at least two different SARS-CoV-2 antigens comprise SARS-CoV-2 structural protein antigens, and/or comprise antigens of the spike (S) protein, membrane (M) protein, nucleocapsid (N) protein, envelope (E) protein, any part thereof, or any combination thereof.

4. (canceled)

5. The vaccine of claim 3, wherein said SARS-CoV-2 spike protein antigen comprises a S1 subunit, S2 subunit, or receptor binding domain (RBD) antigen.

6. (canceled)

7. (canceled)

8. The vaccine of claim 1, wherein said APCs have been contacted with mRNAs encoding SARS-CoV-2 antigens or said APCs have been contacted with a preparation of SARS-CoV-2 particles ex-vivo, wherein:
(a) the preparation of SARS-CoV-2 particles comprises chemically inactivated or attenuated live SARS-CoV-2 particles;
(b) the preparation of SARS-CoV-2 particles comprises at least two SARS-CoV-2 variants; or
(c) both (a) and (b).

9. (canceled)

10. (canceled)

11. (canceled)

12. (canceled)

13. A pharmaceutical composition comprising the vaccine of claim 1.

14. A method for producing a multi-antigenic autologous, cell-based SARS-CoV-2 vaccine, comprising autologous antigen presenting cells (APCs) displaying at least two different SARS-CoV-2 antigens, said method comprising:
(a) obtaining a population of peripheral blood mononuclear cells (PBMCs) from a subject;
(b) isolating and culturing APCs from said population of PBMCs;
(c) contacting said APCs with a preparation of SARS-CoV-2 particles or with mRNAs encoding SARS-CoV-2 antigens for a time period sufficient to generate APCs displaying at least two different SARS-CoV-2 antigens; and
(d) optionally isolating and expanding said APCs from step (c).

15. The method of claim 14, wherein:
(a) the preparation of SARS-CoV-2 particles comprises chemically inactivated or attenuated live SARS-CoV-2 particles;
(b) the preparation of SARS-CoV-2 particles comprises at least two SARS-CoV-2 variants; or
(c) both (a) and (b).

16. (canceled)

17. The method of claim 14, wherein said at least two different SARS-CoV-2 antigens comprise SARS-CoV-2 structural protein antigens, and/or comprise antigens of the spike (S) protein, membrane (M) protein, nucleocapsid (N) protein, envelope (E) protein, any part thereof, or any combination thereof.

18. (canceled)

19. The method of claim 17, wherein said SARS-CoV-2 spike protein antigen comprises a S1 subunit, S2 subunit, or receptor binding domain (RBD) antigen.

20. The method of claim 14, wherein said SARS-CoV-2 particles are contacted at a multiplicity of infection (MOI) of between 0.001 and 10.

21. (canceled)

22. The method of claim 14, wherein the APCs are contacted with a preparation of SARS-CoV-2 particles in the presence of a transduction reagent.

23. The method of claim 22, wherein said transduction reagent comprises polybrene (PB).

24. The method of claim 14, wherein said APCs comprise dendritic cells, macrophages, B cells, monocytes, or a combination thereof.

25. (canceled)

26. A method for preventing or treating a SARS-CoV-2 infection in a subject, the method comprising:
(a) obtaining a population of peripheral blood mononuclear cells (PBMCs) from a subject;
(b) isolating and culturing APCs from said population of PBMCs;
(c) contacting said APCs with a preparation of SARS-CoV-2 particles or with mRNAs encoding SARS-CoV-2 antigens for a time period sufficient to generate APCs displaying at least two different SARS-CoV-2 antigens;
(d) optionally isolating and expanding said APCs from step (c); and
(e) administering said APCs to said subject.

27. The method of claim 26, wherein:
(a) said preparation of SARS-CoV-2 particles comprises chemically inactivated or attenuated live SARS-CoV-2 particles;
(b) the preparation of SARS-CoV-2 particles comprises at least two SARS-CoV-2 variants; or
(c) both (a) and (b).

28. (canceled)

29. The method of claim 26, wherein said at least two different SARS-CoV-2 antigens comprise SARS-CoV-2 structural protein antigens, and/or comprise antigens of the spike (S) protein, membrane (M) protein, nucleocapsid (N) protein, envelope (E) protein, any part thereof, or any combination thereof.

30. (canceled)

31. The method of claim 29, wherein said SARS-CoV-2 spike protein antigen comprises a S1 subunit, S2 subunit, or receptor binding domain (RBD) antigen.

32. The method of claim 26, wherein said SARS-CoV-2 particles are contacted at a multiplicity of infection (MOI) of between 0.001 and 10.

33. (canceled)

34. The method of claim 26, wherein said APCs comprise dendritic cells, macrophages, B cells, monocytes or a combination thereof.

35. (canceled)

36. (canceled)

37. (canceled)
